(19) United States
(12) Patent Application Publication
(10) Pub. No.: US 2004/0132819 A1
(43) Pub. Date: Jul. 8, 2004

(54) Appetite control method

(76) Inventors: Nancy Auestad, Powell, OH (US); Tina D. Wolf, Columbus, OH (US); Yung-Sheng Huang, Upper Arlington, OH (US)

Correspondence Address: ROSS PRODUCTS DIVISION OF ABBOTT LABORATORIES, DEPARTMENT 108140-DS/1, 625 CLEVELAND AVENUE, COLUMBUS, OH, 43215-1724, US

(21) Appl. No.: 10/625,420

(22) Filed: Jul. 23, 2003

Related U.S. Application Data
(63) Continuation-in-part of application No. 10/602,169, filed on Jun. 24, 2003.
(60) Provisional application No. 60/401,466, filed on Aug. 6, 2002.

(51) Int. Cl. A61K 31/202

(52) U.S. Cl. 514/560

(57) ABSTRACT

Products, including nutritional products, dietary supplements and formulas, that contain long chain polyunsaturated fatty acids (LCPs or LC-PUFAs), specifically n-3 LCPs like DHA. Also a methods of using such products to control appetite and help treat and/or prevent obesity and conditions of overweight, especially in a pediatric population. Dietary DHA can act centrally as an antagonist of the CB₁ receptor in the brain in opposition to the endocannabinoids that increase food intake. This is particularly advantageous when DHA is fed during periods of rapid brain growth such as infancy, childhood and adolescence.

This invention relates to products, including nutritional supplements and formulas, that contain long chain polyunsaturated fatty acids (LCPs or LC-PUFAs), specifically n-3 LCPs; and to methods of using such products to control appetite and help treat and/or prevent obesity and conditions of overweight, especially in a pediatric population.

### BACKGROUND

1.1 Introduction

Overweight and obesity have increased markedly in children in Westernized societies in the past decade. Treatment strategies include increasing physical activity and voluntary restriction of calories, in order to affect a negative energy balance. Pharmaceutical interventions have also been attempted. Prevention strategies emphasize balanced nutrition with a regimen of physical activity.

The present invention tests whether the quality of ingested lipids may play a role in regulation of appetite through n-6 and n-3 fatty acyl compounds formed in brain.

Endocannabinoids are a class of naturally occurring compounds that exhibit cannabimimetic properties such as: analgesia, hyperphasia, alteration of cognition and motor control, among other physiological effects including appetite. Within the past decade, endogenous fatty acyl derivatives that to bind to the cannabinoid receptors, better known as CB₁ and CB₂, were discovered. These fatty acyl derivatives are families of compounds, N-acylethanolamines (NAEs) and monoacylglycerols (MAGs; Mechoulam et al, 1998). Arachidonyl ethanolamine, 20:4n-6 NAE, is made up of arachidonic acid and ethanolamine and has recently been shown to increase food consumption when given as an injection to diet-restricted mice and pre-satiated rats (Hao et al, 2001 and Williams and Kirkham, 2001). 20:4n-6 MAG is made up of arachidonic acid and glycerol and has recently been demonstrated to increase food intake when injected into rat brain (Kirkham et al, 2002). Other fatty acyl compounds in the n-6 and n-3 families also bind to the CB receptor, namely those with ≦20 carbons and at least 3 double bonds (Mechoulam et al, 1998).

Arachidonic acid (AA, 20:4n-6) and docosahexaenoic acid (DHA, 22:6n-3) can be made in vivo through the process of desaturation and elongation of the essential fatty acids, linoleic acid and linolenic acid, or obtained from the diet. Studies with animal models during the ‘brain growth spurt’ have shown that varying the levels of dietary essential fatty acids and long-chain n-6 and n-3 polyunsaturated fatty acids results in corresponding changes in the long-chain n-6 and n-3 fatty acids in brain, particularly AA and DHA (Ward et al, 1998 and 1999; de la Presa Owens and Innis, 1999 and 2000). One recent study has demonstrated in formula fed piglets that dietary AA and DHA result in increases in corresponding n-6 and n-3 NAEs and some monoacylglycerols in brain (MAGs; Berger et al, 2001).

It is well established that 20:4n-6 NAE exerts its neurotransmitter-like effects through the cannabinoid receptor, CB₁ (Chaperon and Thiébot, 1999). The CB₁ receptor is found throughout the brain, including the hypothalamus, which is important in appetite regulation.

1.2 Overview of the Literature

1.2.1 Obesity Epidemic

The number of overweight and obese children and adolescents has increased steadily over the past two decades throughout the United States and in many westernized countries (Harnack et al, 2000; Schneider, 2000; Onis and Blössner, 2000; Muller et al, 1999; Heird, 2000; Spruijt-Metz et al, 2002). Overweight is defined by the Centers for Disease Control as an increased body weight in relation to height compared to accepted standards for desirable weight; obesity is defined as an excessive amount of body fat in relation to lean muscle mass (CDC, 2002). Overweight and obesity are more commonly defined as having a body mass index (weight/height²) of between 25 and 29.9 or >30, respectively (CDC, 2002). The prevalence of overweight children (6-17 yrs) in 2000 was between 11-24%, with higher percentages in the older children (Schneider, 2000). Overweight children and adolescents often remain overweight or become obese as adults and are, therefore, at increased risk for comorbidities such as type II diabetes and cardiovascular disease.

Research to identify approaches to prevent childhood obesity is of major public health importance. While many factors contribute to the weight gain that leads to overweight or obesity, most studies have focused on genetic, cultural, behavioral, and environmental factors such as socioeconomic status, sedentary lifestyle, and lack of physical activity. The present research focuses on the central nervous system regulation of food intake.

1.2.2 Central Nervous System Regulation of Food Intake

The central nervous system plays a major role in the regulation of appetite and ultimately food consumption. A healthy weight for adults, as well as children, involves regulation of food intake. This involves balancing energy intake with energy expenditure. When this balance is upset in favor of energy intake, the body is predisposed to store the excess energy. Repeated or prolonged behavior resulting in excess energy storage can lead to becoming overweight, and if sustained, can lead to obesity.

The regulation of food intake is a highly complex process controlled to a large extent by the hypothalamus in the brain. Neural control of energy intake for maintenance of body weight involves a complex integration of neuronal, hormonal, sensory, and thermoregulatory signals from the periphery and within various regions in brain (Williams et al, 2000; Hovel, 2001; van Dijk et al, 2000; Berthoud, 2000).

Some investigators have moved away from studying feeding behavior and satiety per se to studying central nervous system regulation of appetite (Williams et al, 2000; Kaiyala et al, 1995). The hypothalamus plays an important role in the regulation of energy balance. For example, in the hypothalamus increased levels of neuropeptide Y stimulate appetite, increased levels of α-melanocyte-stimulating hormone inhibit feeding and lead to weight loss, and orexin neurones appear to be involved in stimulating feeding in response to low blood glucose levels. Kaiyala et al (1995), who studied central nervous system regulation of energy balance and adiposity, suggest two distinct classes of peripheral signals. Short-term meal related signals and long-term adiposity related signals modulate neuronal pathways in the brain to influence meal initiation and termination.

(1) Leptin and Insulin

Both leptin and insulin are hormones known to provide the brain with information about the amount of fat stored in the body (van Dijk et al, 2000). Thus leptin and insulin help to regulate food intake. Leptin is a peptide hormone secreted from adipose cells. The amount of leptin secreted has been shown to be directly proportional to the amount of fat in storage. Insulin is also a peptide hormone that is secreted from pancreatic B cells and plays a central role in controlling glucose homeostasis and lipid utilization and storage. The amount of insulin secreted at any given time is also directly proportional to the size of body fat stores. Both leptin and insulin act through receptors in the hypothalamus of the brain. The fact that these receptors are found in the hypothalamus provide evidence of direct signals from fat and carbohydrate stores to the brain and suggest a role for these hormones in appetite regulation (Berthoud, 2000). It is has also been reported that higher blood levels of leptin, in the absence of leptin-resistance, have been associated with reduced food intake, and conversely, that lower circulating leptin levels have been associated with increased food intake (Velkoska et al., 2003).

(2) Endocannabinoids

There are many interrelated neuronal pathways, hormones, and receptors involved in the maintenance of not only body weight, but body fat mass as well. Recent research has unveiled fatty acid derived compounds that are formed in the brain and act on a specific receptor known to affect appetite. These compounds are endogenous cannabinoids (i.e. endocannabinoids) and have been shown to play a neuromodulatory role in the regulation of appetite. A brief history of the field is described below, followed by a more detailed description of the two main cannabinoid families, N-acylethanolamines (NAEs) and monoacylglycerols (MAGs).

The active ingredient in cannabis, Δ⁹-tetrahydrocannabinol (THC), has appetite stimulating effects, and is prescribed by some doctors to help patients retain weight (Mechoulam and Fride, 2001). Due to the biological effects of Δ⁹-THC, which are mediated by a specific cannabinoid receptor, referred to as the CB₁ receptor, researchers began to look for endogenous compounds, endocannabinoids. In the early 1990s, a family of bioactive fatty-acyl compounds that exhibited neuromodulator activity at the cannabinoid receptor was identified (Devane et al, 1992; Hanu\check{s} et al, 1993). Later another family was identified, monoacylglycerols or MAGs, that exhibited neuromodulatory activity at cannabinoid receptors (Sugiura et al, 1995; Mechoulam et al, 1995).

Recent research suggests that both of the endocannabinoid families (NAEs and MAGs) are involved in the leptin signaling pathway in the hypothalamus (Di Marzo et al, 2001; Mechoulam and Fride, 2001). Leptin has been shown to inhibit the formation of NAEs and MAGs. In a study by Di Marzo and colleagues (2001), intravenous injection of leptin reduced levels of 20:4n-6 NAE and 20:4n-6 MAG in brain (Di Marzo et al, 2001). These results suggest that interactions between leptin and the endocannabinoids regulate activation of CB₁ receptors in the hypothalamus to regulate food intake.

In the same study, to evaluate the role of leptin in the endocannabinoid system, Di Marzo et al (2001) injected 125 or 250 μg of leptin intravenously into normal Sprague-Dawley rats. Within 30 minutes, hypothalamic levels of 20:4n-6 NAE and 20:4n-6 MAG decreased 40-50% compared to untreated controls. Additionally, obese Zucker rats with defective leptin signaling showed increases in 20:4n-6 MAG levels in the hypothalamus compared to non-obese Zucker control rats. More observations in leptin-deficient mice showed increases in 20:4n-6 MAG or 20:4n-6 NAE or both in the hypothalamus. Thus, leptin appears to play a substantial role in endocannabinoid regulation.

1.2.3 Identification of Endocannabinoids

Research leading to the identification of NAEs as bioactive fatty acids with neuromodulatory activity began nearly a century ago. Excellent reviews of the history of this research are available (e.g. Mechoulam et al, 1998; Di Marzo et al, 1999; Hillard 2000; Onaivi et al, 2002) and the history is not repeated here.

Δ⁹-THC and other synthetic cannabinoid agonists have been shown to bind to specific cannabinoid receptors, typically referred to as CB₁ and CB₂ receptors, and inhibit adenylate cyclase and N-type calcium channels G protein-coupled signaling pathways (Felder et al, 1993). CB₁ receptors are found primarily in the brain, with some mRNA expressed also in the peripheral organs (adrenal gland, heart, lung, prostate, uterus, ovary, testis, bone marrow, thymus, tonsils, and testis). CB₂ receptors have been found in immune system cells (Buckley et al, 1998). McLaughlin et al (1994) studied the development of the cannabinoid receptor in Sprague-Dawley rat pups and found that cannabinoid receptor mRNA is present in rat pups at adult levels as early as postnatal day 3.

Following identification of CB₁ receptors in brain, researchers began to search for the presence of an endogenous ligand in brain. In 1992, Devane et al reported the identity and structure of a natural brain molecule that binds to the cannabinoid receptor (Devane et al, 1992). They found that fractions of porcine brain extracts contained a compound that bound to the CB₁ receptor. They named this compound anandamide, more commonly referred to now as N-arachidonyl ethanolamine (20:4n-6 NAE). They purified 20:4n-6 NAE and tested the cannabimimetic pharmacological activity by measuring the ability to inhibit the twitch response of isolated murine vas deferentia, a standard model to investigate the mode of action of psychotropic agents. The structure of anandamide was determined by mass spectrometry and nuclear magnetic resonance. The chemical name for 20:4n-6 NAE is [5,8,11,14-eicosatetraenamide, (N-2-hydroxyethyl)-(all-Z)]. Ananadamide and its effects are also described in WO 2001/24645 A1 (Nestle, 2001).

Since then, several other fatty acyl compounds that also bind to the cannabinoid receptor have been identified. In 1993, Hanu\check{s} et al identified two other long-chain fatty acyl ethanolamines that bind to the CB₁ receptor, homo-γ-linolenylethanolamide (20:3n-6 NAE) and 7,10,13,16-docosatetraenylethanolamide (22:4n-6 NAE). In 1995, Sugiura et al and Mechoulam et al isolated a different fatty acyl compound, 2-arachidonylglycerol, or 20:4n-6 MAG, from rat brain and canine gut, respectively, with cannabinoid receptor agonist activity. 20:4n-6 MAG has also been shown to bind to the CB₁ and CB₂ receptors and exhibit cannabimimetic activities both in vitro and in vivo. While most of the research on the specific roles of the endocannabinoids that bind to the CB₁-receptor in brain has been associated with 20:4n-6 NAE, other fatty acyl NAEs and MAGs also bind to the CB₁-receptor (Mechoulam et al, 1998) and may play a role in central nervous system regulation of food intake (Di Marzo et al, 2001; Berger et al, 2001; Kirkham et al, 2002).

1.2.4 Tissue Distribution of Endocannabinoids

20:4n-6 NAE has been found in many species including rat, pig, cow, and human, and in many tissues (Schmid et al 1995; Felder et al, 1996; Kondo et al, 1998; Bisogno et al, 1999; Schmid et al, 2000). NAEs have been found in tissues where CB, receptors are found, including brain, kidney, spleen, testis, skin, blood plasma, and uterus. They are present in concentrations ranging from none detected to 29 pmol/g in rat brain (Mechoulam et al, 1998).

Since 20:4n-6 MAG binds to both the CB₁ and CB₂ receptors, it appears also to be a physiologically important and bioactive molecule. It has been found in canine gut, spleen, pancreas, and in brain (Mechoulam et al, 1998; Bisogno et al, 1999; Schmid et al, 2000; Kondo et al, 1998) with concentrations in brain as much as 800 times higher than anandamide (Suguira and Waku, 2000).

1.2.5 Biosynthesis of NAEs and MAGs

The proposed mechanism for NAE biosynthesis involves the Ca²⁺-dependent transfer of a fatty acyl chain from the sn-1 position of a phosphatidylcholine to the primary amine of phosphatidylethanolamine, forming N-acylphosphatidylethanolamine (NAPE) and lyso-phosphatidylcholine (Patricelli and Cravatt 2001). NAPE is subsequently hydrolyzed by a phospholipase D-like enzyme to yield the corresponding NAE and phosphatidic acid. These two reactions are thought to be tightly coordinated.

The proposed mechanism for MAG biosynthesis is similar to that for NAE as has been shown to be Ca²⁺-dependent (Mechoulam et al, 1998). A phosphoinositide-specific phospholipase C causes the release of diacylglycerol and a inositol-triphosphate, which is subsequently hydrolyzed to yield MAG by sn-1-diacylglycerol lipase (Ameri, 1999).

1.2.6 Transport and Degradation of NAEs and MAGs

After release from the phospholipid membrane, NAEs and MAGs are available to bind to the CB1 receptor. They are also hydrolyzed rapidly by a membrane bound enzyme called fatty acyl amide hydrolase (FAAH) or sometimes referred to as ‘anandamide [20:4n-6 NAE] hydrolase’ (Patricelli and Cravatt, 2001; Goparaju et al, 1998; Giang and Cravatt, 1997). Giuffrida et al (2001) have proposed that 20:4n-6 NAE and 20:4n-6 MAG are hydrolyzed by a two-step process involving enzymatic hydrolysis after transport by a specific carrier into the site of degradation. Due to their rapid degradation, endocannabinoids are thought to be formed and used in close proximity to the CB₁ receptor. A carrier-mediated transport of NAEs and MAGs into cells has been proposed based on a fast rate of action, temperature dependence, saturability, and substrate selectivity.

Additional research will be needed to further understand how degradation of NAEs and MAGs is regulated. However most researchers agree that FAAH is the key enzyme involved in hydrolysis of these endocannabinoids. FAAH appears to be a general hydrolytic enzyme, acting on many biologically active lipids and esters (Giuffrida et al, 2001). 20:4n-6 NAE is hydrolyzed to free arachidonic acid and ethanolamine by FAAH. 20:4n-6 MAG is broken down into free arachidonic acid and glycerol through FAAH enzymatic action. Another mechanism of degradation for MAGs has been suggested, possibly a monoacylglycerol lipase, although this has not been firmly established.

1.2.7 Dietary Fatty Acids and Brain Fatty Acid Composition

Studies with formula-fed rats (Ward et al, 1998 and 1999; Wainwright et al, 1999) and piglets (de la Presa Owens and Innis, 1999 and 2000; Arbuckle and Innis, 1993) have shown that feeding different dietary long-chain n-6 and n-3 fatty acids results in differences in the relative amounts of the long-chain n-6 and n-3 fatty acids in brain. Specifically, different amounts and ratios of the dietary essential fatty acids, linoleic acid (18:2n-6) and linolenic acid (18:3n-3), and/or their long-chain polyunsaturated fatty acid derivatives, arachidonic acid (20:4n-6) and docosahexaenoic acid (22:6n-3), respectively, lead to differences in the levels of 20:4n-6 and 22:6n-3 in phospholipid membranes in brain. Differences in 20:4n-6 and 22:6n-3 levels in brain from breast-fed and formula-fed infants who died during the first year of life have also been reported (Farquharson et al, 1995; Makrides et al, 1994).

Ward et al (1998) demonstrated ‘dose’ related effects of feeding varying amounts of 20:4n-6 and 22:6n-3 in a rat milk formula. Rat pups were fed one of three levels of 20:4n-6 and 22:6n-3 (0%, 0.4%, or 2.4% total fatty acids) using a 3×3 design from postnatal day 5 through 18 by gastrostomy tube. The formulas contained what were considered adequate amounts of the essential fatty acids, 10% of total fatty acids as 18:2n-6 and 1% as 18:3n-3. By postnatal day 18 the red blood cell and brain phospholipid membrane fatty acids generally reflected the fatty acid composition of the supplemented formula fed. In addition, when only 20:4n-6 or 22:6n-3 was fed the levels of the n-6 or n-3 long-chain fatty acid not added were lower in red blood cell and brain phospholipids relative to the unsupplemented controls (i.e. supplementation with 20:4n-6 alone led to increases in 20:4n-6 and decreases in 22:6n-3 in brain phospholipid when compared to unsupplemented controls).

In 1999, de la Presa Owens and Innis studied the effects of a diet deficient in essential fatty acids (0.8% total fatty acids as 18:2n-6 and 0.05% as 18:3n-3) with 0% or 0.2% of 20:4n-6 and 0% or 0.16% of 22:6n-3. They fed piglets one of the formulas from birth to postnatal day 18 and found that the supplemented formula increased in 20:4n-6 and 22:6n-3 in brain phospholipid membranes. Piglets fed the diet deficient in essential fatty acids had lower 20:4n-6 and 22:6n-3 when compared to piglets fed adequate essential fatty acids (8.3% 18:2n-6 and 0.8% 18:3n-3).

1.2.8 Dietary Fatty Acids and NAEs and MAGs

Since dietary fatty acids have been shown to influence the levels of 20:4n-6 and 22:6n-3 fatty acids in brain phospholipids, it is reasonable to hypothesize that different n-6 and n-3 dietary fatty acids could lead to similar changes in the brain levels of the bioactives 20:4n-6 NAE and 20:4n-6 MAG. One study with formula-fed piglets (Berger et al, 2001) has provided initial evidence of such an effect. Berger et al produced evidence that different levels of dietary 20:4n-6 and 22:6n-3 fatty acids increased their corresponding NAEs and some MAGs as well as other long-chain fatty acyl NAEs and MAGs. Piglets were fed formulas containing 0.3% 20:4n-6 or 0.2% 22:6n-3, or both 0.3% 20:4n-6 and 0.2% 22:6n-3 during the first 18 days of life. All of the formulas contained adequate levels of essential fatty acids (15-16% 18:2n-6 and 1.5% 18:3n-3 as % total fatty acids). They showed that the piglet diets containing 20:4n-6 and 22:6n-3 yielded increases in the long-chain n-6 and n-3 NAEs and MAGs in brain. 20:4n-6 NAE increased 4-fold, 20:5n-3 NAE increased 5-fold, 22:5n-3 and 22:6n-3 NAE increased 9-10-fold, 22:4n-6 MAG and 22:6n-3 MAG increased nearly 2-fold; whereas 20:4n-6 MAG did not increase. They proposed that dietary fatty acids modulate NAE levels by changing levels of NAE precursors or by providing substrate for biosynthesis.

1.2.9 Injectable NAEs and Food Intake/Appetite Control

There is a growing body of evidence for an association between 20:4n-6 NAE and feeding behavior. Studies of feeding behavior in presatiated rats (Williams and Kirkham, 1999) and fasting mice (Hao et al, 2000) report effects on food intake following injection of 20:4n-6 NAE. Other studies with suckling mouse pups (Fride et al, 2001) and CB₁ receptor knockout mice (Di Marzo et al, 2001) report reduced food intake after administering a CB₁ receptor antagonist (SR141716A) [(N-piperidin-1-yl)-5(4-chlorophenyl)-1-(2,4-dichlorophenyl)-4-methyl-1-H-pyrazole-3-carboxamide].

In pre-satiated rats, Williams and Kirkham (1999) studied whether 20:4n-6 NAE could induce overeating and whether this could be associated by specific action at the CB₁ receptor. During the same study, but in a second series of assessments, 8 rats received a subcutaneous injection of the specific CB₁ receptor antagonist before receiving 1.0 mg/kg injection of 20:4n-6 NAE. All doses of 20:4n-6 NAE induced significant overeating. Overeating that was induced by administering 20:4n-6 NAE was also blocked by CB₁ antagonist pretreatment. The authors suggested that the 20:4n-6 NAE given subcutaneously may have mimicked the actions of an endogenous N-acylethanolamine system involved in the regulation of appetite and that this involved the CB₁ receptor in the hypothalamus.

In a diet-restriction model, Hao et al (2001) studied the effect of low doses of 20:4n-6 NAE (0.001 mg/kg) on food intake response following a 40% calorie restriction. In their study, inbred female BALB/c mice were randomly assigned to vehicle or 20:4n-6 NAE treatment. The mice were given food cakes weighed before and after feeding, including spillage. The mice were fed for 7 days for 2.5 hours per day (between 9 am and 12 pm). Ten minutes before feeding, 0.001, 0.7, or 4 mg/kg of 20:4n-6 NAE in vehicle or vehicle alone was injected intraperitoneally in a volume of 0.1 mL/10 g of body weight. The control group received sufficient calories to maintain weight, whereas the diet restriction group received 40% of the calories given to the control group. Diet restriction was continued until weight plateaued or reached 15 g or less. The study showed that mice injected with 0.001 of mg/kg 20:4n-6 NAE consumed significantly more food than the control group. The 0.001 mg/kg 20:4n-6 NAE treated group also showed improved cognitive function and reversal of most effects of severe food restriction. The other two 20:4n-6 NAE treated groups did not show any significant change. These results suggested that the effect of 20:4n-6 NAE on appetite may be variable depending on the dose and experimental circumstances. The CB, receptor activity appears to be biphasic.

Fride et al (2001) studied the effects of blocking CB, receptor activity in suckling mouse pups. On postnatal day 1 or 2, mice were injected intraperitoneally with 20 mg/kg of a CB₁ receptor antagonist (SR141716A). The researchers observed overwhelming effects on mortality. Injecting the antagonist on postnatal day 1 resulted in death in all rat pups by day 4 and injecting it on postnatal day 2 resulted in death in 50% of the rat pups. In the same study, but a different experiment (Fride et al, 2001), mouse pups were injected with 20 mg/kg of the antagonist daily from postnatal day 2 through day 8. All of the pups immediately stopped gaining weight and died by day 8. Co-administration of Δ⁹-THC with the antagonist led to slight increases in weight gain through day 8. Co-administration of 20:4n-6 MAG with the antagonist did not promote weight gain or extend life. The researchers concluded from these experiments that the endocannabinoid system plays a vital role in milk suckling and growth and development during early stages of mouse life.

In a recent study with CB₁ receptor knockout mice, Di Marzo et al (2001) evaluated leptin and endocannabinoid involvement in the maintenance of food intake. CB₁ receptor knockout mice and wild-type controls were given an injection intraperitoneally of vehicle or the CB₁ receptor antagonist after fasting for 18 hours. CB₁ receptor knockout mice given vehicle ate significantly less than wild-type controls. The CB₁ receptor antagonist decreased food intake in wild-type controls to the level of food intake of the CB, receptor knockout mice given vehicle; administration of the antagonist to the CB₁ receptor knockout mice resulted in no changes in food intake. These results provided further evidence that endocannabinoids may be involved in food intake regulation.

In summary, studies have shown that differences in dietary n-6 and n-3 fatty acids affect brain n-6 and n-3 phospholipid fatty acid composition, and corresponding brain n-6 and n-3 NAEs and MAGs. Further, studies involving injection of 20:4n-6 NAE in rodents have demonstrated effects on appetite and eating behavior.

### 1.3 REFERENCES

Following is an alphabetical list of references that are of interest. A brief description of each is found in the background discussion above or elsewhere in the application.

Ameri A. The effects of cannabinoids on the brain. Progress in Neurobiology, 58:315-348, (1999).

Arbuckle L D and Innis S M. Docosahexaenoic acid is transferred through maternal diet to milk and to tissues of natural milk-fed piglets. J Nutr, 123(10):1668-1675, (1993).

Auestad N, Korsak R A, Bergstrom J D, and Edmond J. Milk-substitutes comparable to rat's milk; their preparation, composition, and impact on development and metabolism in the artificially reared rat. British Journal of Nutrition, 61: 495-518, (1989).

Barinaga M. Pot, Heroin unlock new areas for neuroscience. Science, 258:1882-1884, (1992).

Berger A, Crozier G, Bisogno T, Cavaliere, Innis S, and Di Marzo V. Anandamide and diet: Inclusion of dietary arachidonate and docosahexaenoate leads to increased brain levels of the corresponding N-acylethanolamines in piglets. PNAS, 98(11):6402-6406, (2001).

Berthoud H-R. An overview of neural pathways and networks involved in the control of food intake and selection. In: Neural and Metabolic Control of Macronutrient Intake (H-R Berthoud and R J Seeley, Eds). CRC Press, Boca Raton, Fla. Chapter 24: 361-387, (2000).

Bisogno T, Berrendero F, Ambrosino G, Cebeira M, Ramos J A, Fernandez-Ruiz J J, and Di Marzo V. Brain regional distribution of endocannabinoids: implications for their biosynthesis and biological function. Biochemical and Biophysical Research Communications, 256:377-380, (1999).

Buckley N E, Hansson S, Harta G, and Mezey É. Expression of the CB₁ and CB₂ receptor messenger RNAs during embryonic development in the rat. Neuroscience, 82(4):1131-1149, (1998).

Centers for Disease Control and Prevention. Obesity and Overweight: Basics about overweight and obesity. http://www.cdc.gov/nccdphp/dnpa/obesity/basics.htm (2002)

Chaperon F and Thiébot M-H. Behavioral effects of cannabinoid agents in animals. Critical Reviews in Neurobiology, 13(3):243-281, (1999).

de la Presa Owens S and Innis S M. Docosahexaenoic and arachidonic acid prevent a decrease in dopaminergic and serotoninergic neurotransmitters in frontal cortex caused by a linoleic and α-linolenic acid deficient diet in formula-fed piglets. Journal of Nutrition, 129:2088-2093, (1999).

de la Presa Owens S and Innis S M. Diverse, region-specific effects of addition of arachidonic and docosahexanoic acids to formula with low or adequate linoleic and α-linolenic acids on piglet brain monoaminergic neurotransmitters. Pediatric Research, 48:125-130, (2000).

Devane W A, Hanu\check{s} L, Breuer A, Pertwee R G, Stevenson L A, Griffin G, Gibson D, Mandelbaum A, Etinger A, and Mechoulam R. Isolation and structure of a brain constituent that binds to the cannabinoid receptor. Science, 258:1946-1949, (1992).

Di Marzo V, De Petrocellis L, Bisogno T, and Melck D. Metabolism of anandamide and 2-arachidonoylglycerol: an historical overview and some recent developments. Lipids, 34:S319-S325, (1999).

Di Marzo V, Goparaju S K, Wang L, Liu J, Bátkal S, Jaral Z, Fezza F, Miura G I, Palmiter R D, Sugiura T, and Kunos G. Leptin-regulated endocannabinoids are involved in maintaining food intake. Nature, 410:822-825, (2001).

Farquharson J, Jamieson E C, Abbasi K A, Patrick W J, Logan R W, and Cockburn F. Effect of diet on the fatty acid composition of the major phospholipids of infant cerebral cortex. Arch Dis Child, 72(3):198-203, (1995).

Felder C C, Briley E M, Axelrod J, Simpson J T, Mackie K, and Devane W A. Anandamide, an endogenous cannabimimetic eicosanoid, binds to the cloned human cannabinoid receptor and stimulates receptor-mediated signal transduction. Proc. Natl. Acad. Sci., 90:7656-7660, (1993).

Felder C C, Nielsen A, Briley E M, Palkovits M, Priller J, Axelrod J, Nguyen D N, Richardson J M, Riggin R M, Koppel G A, Paul S M, Becker G W. Isolation and measurement of the endogenous cannabinoid receptor agonist, anandamide, in brain and peripheral tissues of human and rat. FEBS Letters, 393:231-235, (1996).

Folch J, Lees M, and Sloane-Stanley G A. J. Biol. Chem. 226:497, (1957).

Fontana A, Di Marzo V, Cadas H, and Piomelli D. Analysis of anandamide, an endogenous cannabinoid substance, and of other natural N-acylethanolamines. Prostaglandins Leukotrienes and Essential Fatty Acids, 53:301-308, (1995).

Fride E, Ginzburg Y, Breuer A, Bisogno T, Di Marzo V, Mechoulam R. Critical role of the endogenous cannabinoid system in mouse pup suckling and growth. European Journal of Pharmacology, 419:207-214, (2001).

Gaoni Y and Mechoulam R. Isolation, structure and partial synthesis of an active constituent of hashish. J Am Chem Soc. 86:1646 (1964).

Giang D K and Cravatt B F. Molecular characterization of human and mouse fatty acid amide hydrolases. Proc. Natl. Acad. Sci., 94:2238-2242, (1997).

Giuffrida A, Beltramo M, and Piomelli D. Mechanisms of endocannabinoid inactivation: biochemistry and pharmacology. Journal of Pharmacology and Experimental Therapeutics, 298(1):7-14, (2001).

Goparaju S K, Ueda N, Yamaguchi H, and Yamamoto S. Anandamide amidohydrolase reacting with 2-arachidonoylglycerol, another cannabinoid receptor ligand. FEBS Letters, 422:69-73, (1998).

Guzmán M and Sánchez C. Effects of cannabinoids on energy metabolism. Life Sciences, 65(6/7):657-664, (1999).

Hanu\check{s} L, Gopher A, Almog S, and Mechoulam R. Two new unsaturated fatty acid ethanolamides in brain that bind to the cannabinoid receptor. J. Med. Chem., 36:3032-3034, (1993).

Hao S, Avraham Y, Mechoulam, and Berry E M. Low dose anandamide affects food intake, cognitive function, neurotransmitter and corticosterone levels in diet-restricted mice. European Journal of Pharmacology, 392:147-156, (2000).

Harnack L J, Jeffrey R W, and Boutelle K N. Temporal trends in energy intake in the United States: an ecological perspective. Am J Clin Nutr, 71:1478-1484, (2000).

Havel P J. Peripheral signals conveying metabolic information to the brain: short-term and long-term regulation of food intake and energy homeostasis. Exp Bio Med, 226(11):963-977, (2001).

Heird W C. Parental feeding behavior and children's fat mass. Am J Clin Nutr, 75:451-452, (2002).

Hillard C J. Biochemistry and pharmacology of the endocannabinoids arachidonylethanolamide and 2-arachidonylglycerol. Prostaglandins and other Lipid Mediators, 61:3-18, (2000).

Kaiyala K J, Woods S C, and Schwartz M W. New model for the regulation of energy balance and adiposity by the central nervous system. Am J Clin Nutr, 62(suppl): 1223s⁻¹²³⁴S, (1995).

Kempe K, Hsu F-F, Bohrer A, and Turk J. Isotope dilution mass spectrometric measurements indicate that arachidonylethanolamine, the proposed endogenous ligand of the cannabinoid receptor, accumulates in rat brain tissue post mortem but is contained at low levels in or is absent from fresh tissue. Journal of Biological Chemistry, 271(29):17287-17295, (1996).

Kirkham T C, Williams C M, Fezza F, and Di Marzo V. Endocannabinoid levels in rat limbic forebrain and hypothalamus in relation to fasting, feeding, and satiation: stimulation of eating by 2-arachidonoyl glycerol. British Journal of Pharmacology, 136:550-557, (2002).

Kondo S, Kondo H, Nakane S, Kodaka T, Tokumura A, Waku K, and Sugiura T. 2-Arachidonoylglycerol, an endogenous cannabinoid receptor agonist: identification as one of the major species on monoacylglycerols in various rat tissues, and evidence for its generation through Ca²⁺-dependent and independent mechanisms. FEBS Letters, 429:152-156, (1998).

Liu J, Lee T, Bobik, Jr. E, Guzman-Harty M, and Hastilow C. Quantitative Determination of Monoglycerides and Diglycerides by High-Performance Liquid Chromatography and Evaporative Light-Scattering Detection. Journal of the American Oil Chemists' Society, 70(4):343-347, (1993).

Makrides M, Neumann M A, Byrad R W, Simmer K, and Gibson R A. Fatty acid composition of brain, retina, and erythrocytes in breast- and formula-fed infants. Am J Clin Nutr, 60(2):189-194, (1994).

McLaughlin C R, Martin B R, Compton D R, and Abood M E. Cannabinoid receptors in developing rats: detection of mRNA and receptor binding. Drug and Alcohol Dependence, 36:27-31, (1994).

Mechoulam R, Ben-Shabat S, Hanus L, Ligumski L, Kaminski N E, Schatz A R, Gopher A, Amlog S, Martin B R, and Comton D R. Identification of an endogenous 2-monoglyceride, present in canine gut, that binds to cannabinoid receptors. Biochem Pharmacol, 50:83-90, (1995).

Mechoulam R and Fride E. A hunger for cannabinoids. Nature, 410:763-764, (2001).

Mechoulam R, Fride E, and Di Marzo V. Endocannabinoids. European Journal of Pharmacology, 359:1-18, (1998).

Müller M J, Koertringer I, Mast M, Languix, and Frunch A. Physical activity and diet in 5 to 7 years old children. Public Health Nutrition, 2(3a):443-444, (1999).

Onaivi E S, Leonard C M, Ishiguro H, Zhang P W, Lin Z, Akinshola B E, and Uhl G R. Endocannabinoids and cannabinoid receptor genetics. Progress in Neurobiology, 66:307-344, (2002).

Onis M de and Blössner M. Prevalence and trends of overweight among preschool children in developing countries. Am J Clin Nutr, 72:1032-1039, (2000).

Patricelli M P and Cravatt B F. Proteins regulating the biosynthesis and inactivation of neuromodulatory fatty acid amides. Vitamins and Hormones, 62:95-131, (2001).

Reeves P G, Nielsen F H, and Fahey G C, Jr. AIN-93 purified diets for laboratory rodents: final report of the American Institute of Nutrition ad hoc writing committee on the reformulation of the AIN-76A rodent diet. Journal of Nutrition, 123:1939-1951, (1993).

Salzet M, Breton C, Bisogno T, and Di Marzo V. Comparative biology of the endocannabinoid system—possible role in the immune response. Eur J Biochem, 267:4917-4927, (2000).

Schmid P C, Krebsbach R J, Perry S R, Dettmer T M, Maasson J L, and Schmid H H O. Occurrence and postmortem generation of anandamide and other long-chain N-acylethanolamines in mammalian brain. FEBS Letters, 375:117-120, (1995).

Schmid P C, Schwartz K D, Smith C N, Krebsbach R J, Berdyshev E V, and Schmid H H O. A sensitive endocannabinoid assay. Th esimultaneous analysis of N-acylethanolamines and 2-monoacylglycerols. Chemistry and Physics of Lipids, 104:185-191, (2000).

Schneider D. International trends in adolescent nutrition. Social Science & Medicine, 51:955-967 (2000).

Smart J L, Stephens D N, and Katz H B. Growth and development of rats artificially reared on a high or a low plane of nutrition. British Journal of Nutrition, 49: 497-506, (1983).

Smart J L, Stephens D N, Tonkiss J, Auestad N S, and Edmond J. Growth and development of rats artificially reared on different milk-substitutes. British Journal of Nutrition, 52: 227-237, (1984).

Sonnenberg N, Bergstrom J D, Ha Y H, and Edmond J. Metabolism in the artificially reared rat pup: effect of an a typical rat milk substitute. Journal of Nutrition, 112: 1506-1514, (1982).

Spruijt-Metz D, Lindquist C H, Birch L L, Fisher J O, and Goran M I. Relation between mothers' child-feeding practices and children's adiposity. Am J Clin Nutr, 75:581-586, (2002)

Sugiura T, Kodaka T, Nakane S, Kishimoto A, Konda S, and Waku K. 2-Arachidonylglycerol: a possible endogenous cannabinoid ligand in brain. Biochem. Biophys. Res. Commun, 215:89-97, (1995).

Sugiura T and Waku K. 2-Arachidonylglycerol: a possible multifunctional lipid mediator in the nervous and immune systems. Annals New York Academy of Sciences, VOL: 344-346, (2000).

Van Dijk G, Chavez M, Riedy C A, and Woods S C. Adiposity signals and macronutrient selection. In: Neural and Metabolic Control of Macronutrient Intake. (H-R Berthoud and RJ Seeley, Eds). CRC Press, Boca Raton, Fla. Chapter 30: 465-472, (2000).

Velkoska E, et al. Leptin reduces food intake but does not alter weight regain following food deprivation in the rat. Int J Obes Relat Metab Disord 27:48-54 (2003)

Wang Y, Liu Y, Ito Y, Hashiguchi T, Kitajima I, Yamakuchi M, Shimizu H, Matsuo S, Imaizumi H, and Maruyama I. Simultaneous measurement of anandamide and 2-arachidonoylglycerol by polymyxin B-selective adsorption and subsequent high performance liquid chromatography analysis: increase in endogenous cannabinoids in the sera of patients with endotoxic shock. Analytical Biochemistry, 294:73-82, (2001).

Ward G R, Huang Y S, Bobik E, Xing H-C, Mutsaers L, Auestad N, Montalto M, and Wainwright P. Long-chain polyunsaturated fatty acid levels in formulae influence deposition of docosahexaenoic acid and arachidonic acid in brain and red blood cells of artificially reared neonatal rats. Journal of Nutrition, 128: 2473-2487, (1998).

Ward G R, Huang Y-S, Xing H-C, Bobik E, Wauben I, Auestad N, Montalto M, and

Wainwright P E. Effects of γ-linolenic acid and docosahexaenoic acid in formulae on brain fatty acid composition in artificially reared rats. Lipids, 34: 1057-1063, (1999).

Wainwright P E, Xing H-C, Ward G R, Huang Y-S, Bobik E, Auestad N, and Montalto M. Water maze performance is unaffected in artificially reared rats fed diets supplemented with arachidonic acid and docosahexaenoic acid. Journal of Nutrition, 129: 1079-1089, (1999).

Williams C M and Kirkham T C. Anandamide induces overeating: mediation by central cannabinoid (CB₁) receptors. Psychopharmacology, 143:315-317, (1999).

Williams G, Harrold J A, and Cutler D J. The hypothalamus and the regulation of energy homeostasis: lifting the lid on a black box. Proceedings of the Nutritional Society, 59:385-396, (2000).

### SUMMARY OF THE INVENTION

There are several aspects of the present invention. In a first aspect, the invention comprises a method for decreasing the appetite of a mammal comprising enterally administering to said mammal an amount of long-chain n-3 PUFA effective to decrease the appetite of said mammal.

In a second aspect, the invention comprises a method for antagonizing the CB₁ receptor in the brain of a mammal comprising enterally administering to said mammal an amount of long-chain n-3 PUFA effective to antagonize the CB₁ receptor activity in the brain of said mammal.

In a third aspect, the invention comprises a method for decreasing the incidence of obesity or overweight status in a population of mammals comprising enterally administering to at least some members of said population an amount of long-chain n-3 PUFA effective to modulate negatively the appetite of said mammal.

In a fourth aspect, the invention comprises a method for increasing serum leptin levels in humans or other mammals, preferably to reduce appetite as a result of, in whole or in part, the serum leptin level increase, more preferably to also reduce the incidence or extent of obesity in such humans or other mammals. The leptin level increase is preferably determined by postprandial or fasting serum measurement following administration of a DHA-containing nutritional composition (or other long chain n-3 PUFA-containing composition) relative to a similar measurement following administration of a similar nutritional composition but without DHA (or other similar long chain n-3 PUFA). In this particular aspect of the present invention, the long-chain n-3 PUFA preferably comprises DHA, and more preferably is administered to a child or adult in a daily amount of from about 84 to about 15,832 mg.

In each of these aspects, a preferred long-chain n-3 PUFA is DHA; and this may be administered independent of AA. Preferably, the long-chain n-3 PUFA is administered during a growth phase. Preferably, the long-chain n-3 PUFA is administered prior to or in conjunction with an appetite-impacting stimulus. In each aspect, the preferred effective dosing levels are about 8 to about 396 mg/kg/day for an infant, (preferably about 127 to 165 mg/kg/day); about 84 to about 11610 mg/day for a child up to age 15 and about 84 to about 15,832 mg/day for an adult. More preferred levels are included herein.

In a final aspect, the invention comprises a method for modulating the appetite of a mammal comprising enterally administering to said mammal an amount of long-chain n-3 PUFA and an amount of long-chain n-6 PUFA in relative amounts effective to modulate the appetite of said mammal. The long-chain n-3 PUFA preferably comprises DHA and the long-chain n-6 PUFA preferably comprises AA. Preferably, the long-chain n-3 PUFA is administered during a growth phase. Preferably, the long-chain n-3 PUFA is administered prior to or in conjunction with an appetite-impacting stimulus. In each aspect, the preferred effective dosing levels are about 8 to about 396 mg/kg/day for an infant, (preferably about 127 to 165 mg/kg/day); about 84 to about 11610 mg/day for a child up to age 15 and about 84 to about 15,832 mg/day for an adult. More preferred levels are included herein.

### DETAILED DESCRIPTION

2.1 Lipid Terminology

Fatty acids are an important component of nutrition. Fatty acids are carboxylic acids and are classified based on the length and saturation characteristics of the carbon chain. Long chain fatty acids have from 16 to 24 or more carbons and may also be saturated or unsaturated. In longer fatty acids there may be one or more points of unsaturation, giving rise to the terms “monounsaturated” and “polyunsaturated”, respectively. Long chain polyunsaturated fatty acids, (LCP's or LC-PUFAs) having 20 or more carbons are of particular interest in the present invention.

LC-PUFAs are categorized according to the number and position of double bonds in the fatty acids according to a nomenclature well understood by the biochemist. There are two main series or families of LC-PUFAs, depending on the position of the double bond closest to the methyl end of the fatty acid: the n-3 series contains a double bond at the third carbon, while the n-6 series has no double bond until the sixth carbon. Thus, arachidonic acid (“AA” or “ARA”) has a chain length of 20 carbons and 4 double bonds beginning at the sixth carbon. As a result, it is referred to as “20:4 n-6”. Similarly, docosahexaenoic acid (“DHA”) has a chain length of 22 carbons with 6 double bonds beginning with the third carbon from the methyl end and is thus designated “22:6 n-3”. AA and DHA are of particular importance in the present invention.

Other important LCPs are the C18 fatty acids that are precursors in these biosynthetic pathways, as is described in U.S. Pat. No. 5,223,285. Thus it is known that 110 linoleic (118:2n-6, “LA”) and intermediates γ-linolenic (118:3n-6, “GLA”) and dihomo-γ-linolenic (20:3n-6, “DHGLA”) are important precursors to AA (20:4n-6). Similarly, α-linolenic (118:3n-3, “ALA”) and intermediates stearodonic (118:4n-3) and EPA (20:5n-3) are important precursors to DHA (22:6n-3).

Fatty acids are often found in nature as acyl radicals esterified to alcohols. A glyceride is such an ester of one or more fatty acids with glycerol (1,2,3-propanetriol). If only one position of the glycerol backbone molecule is esterified with a fatty acid, a “4monoglyceride” is produced; if two positions are esterified, a “diglyceride” is produced; and if all three positions of the glycerol are esterified with fatty acid a “triglyceride” or “triacylglycerol” is produced.

A phospholipid is a special type of diglyceride, wherein the third position on the glycerol backbone is bonded to a nitrogen containing compound such as choline, serine, ethanolamine, inositol, etc., via a phosphate ester. Triglycerides and phospholipids are often classified as long chain or medium chain, according to the fatty acids attached thereto. A “source” of fatty acids may include any of these forms of glycerides from natural or other origins.

“Lipid” is a general term describing fatty or oily components. In nutrition, lipids provide energy and essential fatty acids and enhance absorption of fat soluble vitamins. The type of lipid consumed affects many physiological parameters such as plasma lipid profile, cell membrane and organ lipid composition and synthesis of mediators of the immune response such as prostaglandins and thromboxanes. Other physiological effects of lipids are described in the background.

Sources of longer LCPs include dairy products like eggs and butterfat; marine oils, such as cod, menhaden, sardine, tuna and many other fish; certain animal fats, lard, tallow and microbial oils such as fungal and algal oils as described in detail in U.S. Pat. Nos. 5,374,657, 5,550,156, and 5,658,767. Notably, fish oils are a good source of DHA and they are commercially available in “high EPA” and “low EPA” varieties, the latter having a high DHA:EPA ratio, preferably at least 3:1. Algal oils such as those from dinoflagellates of the class Dinophyceae, notably Crypthecodinium cohnii are also sources of DHA (including DHASCO™), as taught in U.S. Pat. Nos. 5,397,591, 5,407,957, 5,492,938, and 5,711,983. The genus Mortierella, especially M. alpina, and Pythium insidiosum are good sources of AA, including ARASCO™ as taught by U.S. Pat. No. 5,658,767 and as taught by Yamada, et al. J. Dispersion Science and Technology, 10(4&5), pp561-579 (1989), and Shinmen, et al. Appl. Microbiol. Biotechnol. 31:11-16 (1989).

Of course, new sources of LCPs may be developed synthetically or through the genetic manipulation of other organisms, particularly vegetables and/or oil bearing plants. Desaturase and elongase genes have been identified from many organisms and these might be engineered into plant or other host cells to cause them to produce large quantities of LCP-containing oils at low cost. The use of such synthetic or recombinant oils are also contemplated in the present invention.

2.2 Stimulation or Stress

In one aspect, the present invention is utilized in combination with an environmental stress or stimulus. Studies in rodents have shown that mild to moderate stressors result in increased food intake, while a more severe stress does not (Harris et al 2000). The effect of stress on food intake depends on the duration of the stressor and includes both physical and psychological stressors. Mild stressors known to elicit increased food intake in rats include tail pinch, a brief period of restraint or handling, food restriction, and sleep deprivation.

Children in Westernized societies experience intermittent mild stressors, which by inference may elicit an appetitive response. Examples may include irregular meal times, sleep deprivation (Sekine et al 2002; Buboltz et al 2001), and parental expectations to excel in school and/or sports. Children who are latch-key kids are likely to encounter additional intermittent stressors. Stressors or stimuli that have the effect of increasing food intake (i.e. eliciting an appetitive response) are referred to herein as “appetite-impacting” stressors or stimuli.

The food restriction periods in the present study represent such mild stressors that elicited an appetitive response. This was most apparent following the overnight 40% food restriction period on day 19, and less so following the overnight fast on day 20. The differences in appetitive response following the different food restriction paradigms may be explained by limited sample size, an adaptive response to the fasting/feeding paradigm, or the latter (overnight fast) exceeded a mild/moderate stress threshold.

2.3 Product Forms

The dietary fatty acids of the present invention may be given in many forms, including but not limited to, nutritional products, dietary supplements, pharmaceuticals or other products. They may be used at any age, for example by infants, children or adults. There may be particular value in using them during periods of rapid growth, such as infancy, childhood and adolescence. The dietary fatty acids of the invention may be incorporated into a nutritious “vehicle or carrier” which includes but is not limited to the FDA statutory food categories: conventional foods, foods for special dietary uses, dietary supplements and medical foods.

2.3.1 Nutritional Products

Nutritional products contain macronutrients, ie. fats, proteins and carbohydrates, in varying relative amounts depending on the age and condition of the intended user, and often contain micronutrients such as vitamins, minerals and trace minerals. The term “nutritional product” includes but is not limited to these FDA statutory food categories: conventional foods, foods for special dietary uses, medical foods and infant formulas. “Foods for special dietary uses” are intended to supply a special dietary need that exists by reason of a physical, physiological, pathological condition by supplying nutrients to supplement the diet or as the sole item of the diet. A “medical food” is a food which is formulated to be consumed or administered enterally under the supervision of a physician and which is intended for the specific dietary management of a disease or condition for which distinctive nutritional requirements, based on recognized scientific principles, are established by medical evaluation.

In addition, a “dietary supplement” is a product intended to supplement the diet by ingestion in tablet, capsule or liquid form and is not represented for use as a conventional food or as a sole item of a meal or the diet.

2.3.2 Infant Formulas

Infant formula refers to nutritional formulations that meet the standards and criteria of the Infant Formula Act, (21 USC §350(a) et. seq.) and are intended to replace or supplement human breast milk. Although such formulas are available in at least three distinct forms (powder, liquid concentrate and liquid ready-to-feed (“RTF”), it is conventional to speak of the nutrient concentrations on an “as fed” basis and therefore the RTF is often described, it being understood that the other forms reconstitute or dilute according to manufacturer's directions to essentially the same composition and that one skilled in the art can calculate the relevant composition for concentrated or powder forms.

“Standard” or “Term” infant formula refers to infant formula intended for infants that are born full term as a first feeding. The protein, fat and carbohydrate components provide, respectively, from about 8 to 10, 46 to 50 and 41 to 44% of the calories; and the caloric density ranges narrowly from about 660 to about 700 kcal/L (or 19-21 Cal/fl.oz.), usually about 675 to 680 (20 Cal/fl.oz.). The distribution of calories among the fat, protein and carbohydrate components may vary somewhat among different manufacturers of term infant formula. SIMILAC™ (Ross Products Division, Abbott Laboratories), ENFAMIL™ (Mead Johnson Nutritionals), and GOOD START™ (Carnation) are examples of term infant formula.

“Nutrient-enriched” formula refers to infant formula that is fortified relative to “standard” or “term” formula. The primary defining characteristic that differentiates nutrient-enriched formulas is the caloric density; a secondary factor is the concentration of protein. For example, a formula with a caloric density above about 700 Kcal/L or a protein concentration above about 18 g/L would be considered “nutrient-enriched”. Nutrient-enriched formulas typically also contain higher levels of calcium (e.g. above about 650 mg/L) and/or phosphorus (e.g. above about 450 mg/L). Examples include Similac NEOSURE™ and Similac Special Care™ formulas.

2.3.3 Dietary Supplements

Dietary supplements are soft gels, capsules, powders, tablets, liquids and other dosage forms with specific nutrients that are generally intended to support the normal structure and function of the body. Dietary supplements may be formulated with suitable excipients and carriers, much like standard pharmaceutical products.

Soft gels are widely used in the pharmaceutical industry as an oral dosage form containing many different types of pharmaceutical and vitamin products. Soft gels are available in a great variety of sizes and shapes, including round shapes, oval shapes, oblong shapes, tube shapes and other special types of shapes such as stars. The finished capsules or soft gels can be made in a variety of colors, with or without opacifiers. Soft gels are predominantly employed for enclosing liquids, more particularly oily solutions, suspensions or emulsions. Filling materials normally used are vegetable, animal or mineral oils, liquid hydrocarbons, volatile oils and polyethylene glycols.

The soft gelatin capsules can be manufactured using techniques well known to those skilled in the art. U.S. Pat. Nos. 4,935,243, 4,817,367 and 4,744,988 are directed to the manufacturing of soft gelatin capsules. Manufacturing variations are certainly well known to those skilled in the pharmaceutical sciences. Typically, these comprise an outer shell primarily made of gelatin, a plasticizer, and water, and a fill contained within the shell. The fill may be selected from any of a wide variety of substances that are compatible with the gelatin shell.

Generally speaking, a gelatin capsule manufacturing system is comprised of three main systems: a sheet forming unit, a capsule forming unit, and a capsule recovery unit. Melted gelatin is formed into sheets of desired thickness which is inserted between a pair of die rolls fitted with the desired die heads in the capsule-forming unit. For liquid-filled capsules, a fill nozzle is positioned so as to discharge the desired amount of fill liquid between two gelatin sheets. The discharging timing is adjusted so that the recess formed by the die heads are filed with fill liquid as the gelatin sheets are brought into contact with each other, which allows filled capsules to be formed. Die roll scraping brushes remove the formed gelatin capsules from the die heads. The gelatin capsules are subsequently collected into a bulk container for storage prior to filing into the desired container. Fro dry-filled capsules, the two halves of the shell may be formed separately and sealed after filling.

Tablets are generally formed by compression of the active ingredient, often as a “pharmaceutically acceptable salt”, along with binders, lubricants and other excipients in a die and mold. Additional details of capsule and tablet formation can be obtained in any of several texts on this topic, including Remington's Pharmaceutical Sciences, XV edition (1975).

Pharmaceutically acceptable salts are well-known in the art. For example, S. M. Berge, et al. describes pharmaceutically acceptable salts in detail in J. Pharmaceutical Sciences, 1977, 66: 1 et seq., which is hereby incorporated herein by reference. The salts may be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quarternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of the compounds by reacting a carboxylic acid-containing moiety with a suitable base such as the hydroxide, carbonate or bicarbonate of a pharmaceutically acceptable metal action or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

2.4 Dosing

The level of a particular fatty acid in a formula is typically expressed as percent of the total fatty acids. This percentage multiplied by the absolute concentration of total fatty acids in the formula (either as g/L or g/100 kcal) gives the absolute concentration of the fatty acid of interest (in g/L or g/100 kcal, respectively). Total fatty acids may be estimated as about 95% of total fat to account for the weight of the glycerol backbone. Conversion from mg/100 kcal to mg/L is a simple calculation dependant on the caloric density as is known to those skilled in the art.

Nutritional compositions enriched in DHA according to the invention may provide from 100%, in the case of a sole source feeding such as infant formula, to less than about 5% of daily caloric intake, in the case of a conventional snack food. If formula is fed to newborns it may be complemented with some human milk. And as the infant gets to about 2-4 months, solid foods often begin to supply some of the calories and the amount of formula may decrease as a percent of total caloric intake. Any nutritive or caloric component of supplements or pharmaceuticals is usually de minimis and disregarded.

It may be beneficial, in accordance with the present invention, to combine the DHA dosing with a mild stressor as noted above.

In the rat pup study example, rats fed DHA at 2.5% of total fatty acids, independent of the level of ARA (0% or 2.5%) with marginal levels of linoleic acid and alpha-linolenic acid during the brain growth spurt ate about 11% less of the weaning diet in the first 2 hr after a food restriction period, a mild appetite-impacting stressor. Others have shown dose related effects of different dietary levels of ARA (0, 0.4%, and 2.3% total fatty acids) and DHA (0, 0.4%, and 2.3%) on brain long-chain n-6 and n-3 fatty acids using the same rat milk formula model (Ward et al 1999).

The relative differences in the 22:6n-3 levels in brain from rats fed the 0 and 2.5% DHA diets in the present study were similar to those reported by Ward et al (1999) for the 0 and 2.3% DHA diets. Based on the association between brain levels of DHA and appetite in the example and the relationship between dietary DHA and the level of DHA in brain (present example and Ward et al 1999), it is reasonable to anticipate about a 5% decrease in food intake with 0.4% dietary DHA. From the public health perspective, a sustained 5% reduction in caloric intake in the population has the potential to reduce the risk of becoming overweight and obese.

TABLE A
Levels of DHA (as % total fatty acids ingested

| | Nutritional Products | | |
|---|---|---|---|
| Designed | | | More |
| for | Range | Preferred | Preferred |
| INFANTS | | | |
| Preterm | 0.10-2.5 | 0.10-1.0 | 0.15-0.50 |
| At birth | 0.10-2.5 | 0.10-1.0 | 0.15-0.50 |
| 2-6 mos | 0.10-2.5 | 0.10-1.0 | 0.15-0.50 |
| 6-12 mos | 0.10-3.0 | 0.10-1.3 | 0.15-0.70 |
| CHILDREN | | | |
| 1-5 years | 0.10-5.0 | 0.10-2.0 | 0.30-1.00 |
| 5-15 years | 0.10-5.0 | 0.10-2.0 | 0.30-1.00 |
| ADULTS | | | |
| adult | 0.10-5.0 | 0.10-2.0 | 0.30-1.00 |

The preferred time to feed the DHA enriched diets is when accretion of the long-chain n-6 and n-3 fatty acids is the fastest—i.e. during infancy, childhood and adolescence. The most rapid rate of brain growth occurs in infancy. However, brain growth and neuronal maturation continues until about 12-20 years of age The fatty acid content in adult brains also can be affected by diet in adults but over a longer time frame. Table B, below gives ranges and preferred ranges for effective dosing of DHA in accordance with the invention. The effective dose of the ingredient, DHA, does not differ whether given as part of a nutritional product, as a supplement or as a pharmaceutical.

TABLE B
Dietary DHA

| | | | | |
|---|---|---|---|---|
| Preferred Intakes | | most | | Daily |
| by Age Group | range preferred | preferred | assuming | Calories |

INFANTS

| Preterm | mg/kg/day | | | kcal/kg/day |
|---|---|---|---|---|
| usual | 13 | 13 | 19 | 120 |
| range lower | 8 | 8 | 13 | 90 |
| upper | 396 | 158 | 79 | 150 |
| Birth to 6 mos | mg/kg/day | | | kcal/kg/day |
| usual | 11 | 11 | 16 | 100 |
| range lower | 8 | 8 | 13 | 80 |
| upper | 317 | 127 | 63 | 120 |
| 6-12 mos | mg/kg/day | | | kcal/kg/day |
| usual | 11 | 11 | 16 | 100 |
| range lower | 8 | 8 | 13 | 80 |
| upper | 380 | 165 | 89 | 120 |
| CHILDREN | | | | |
| 1-5 years | mg/day | | | kcal/day |
| usual | 137 | 137 | 412 | 1300 |
| range lower | 84 | 84 | 253 | 800 |
| upper | 9499 | 3800 | 1900 | 1800 |
| 5-15 years | mg/day | | | kcal/day |
| usual | 190 | 190 | 570 | 1800 |
| range lower | 84 | 84 | 253 | 800 |
| upper | 11610 | 4644 | 2322 | 2200 |
| ADULTS | | | | |
| Adult | mg/day | | | kcal/day |
| usual | 211 | 211 | 633 | 2000 |
| range lower | 84 | 84 | 253 | 800 |
| upper | 15832 | 6333 | 3166 | 3000 |

Thus, for example, a range of effective dosing for a child age 1-5 years is 84 to 9499 mg per day, preferably 84 to 3800 mg per day and most preferably 253 to 2322 mg per day. Comparable values for an adult are 84-15832, preferably 84-6333, most preferably 253-3166. Note that values are given in mg/day for children and adults, and in mg/kg/day for infants. Thus, comparable values for an infant up to about 6 months of age are: 8-380 mg/kg/day, preferably 8-165 mg/kg/day, and most preferably 13-89 mg/kg/day. Throughout this application, numerical ranges given as “x-y” should be interpreted as “from about x to about y”; it being understood that “about” modifies both the value x and the value y. Additionally, such a range is understood to indicate that an infinite number of values between x and y are implicitly and unambiguously disclosed by such range. For example, 0.10-2.5 expressly discloses such values as 0.19, 0.5, 0.823, 1.25, 1.64, 1.999, etc. as well as values that are “about” 0.10 or “about” 2.5.

2.5 Process of Manufacture

The liquid and powder nutritional products of the present invention can be manufactured by generally conventional techniques known to those skilled in the art. Briefly, three slurries are prepared, blended together, heat treated, standardized, spray dried (if applicable), packaged and sterilized (if applicable).

2.5.1 Liquid Products

A carbohydrate/mineral slurry is prepared by first heating water to an elevated temperature with agitation. Minerals are then added. Minerals may include, but are not limited to, sodium citrate, sodium chloride, potassium citrate, potassium chloride, magnesium chloride, tricalcium phosphate, calcium carbonate, potassium iodide and trace mineral premix. A carbohydrate source, such as one or more of lactose, corn syrup solids, sucrose and/or maltodextrin is dissolved in the water, thereby forming a carbohydrate solution. A source of dietary fiber, such as soy polysaccharide, may also be added. The completed carbohydrate/mineral slurry is held under agitation at elevated temperature until it is blended with the other slurries, preferably for no longer than about twelve hours.

An oil slurry is prepared by combining and heating the basic oil blend. The basic oil blend typically contains some combination of soy, coconut, palm olein, high oleic safflower or sunflower oil and medium chain triglycerides. Emulsifiers, such as diacetyl tartaric acid esters of mono, diglycerides, soy mono, diglycerides, and soy lecithin may be used. Any or all of the oil-soluble vitamins A, D, E (natural R,R,R form or synthetic) and K may be added individually or as part of a premix. Beta carotene, which can function as an in vivo antioxidant, may also be added, as may a stabilizer such as carrageenan. Oils containing specific LCPs important to this invention (e.g. DHA and AA) can be added to the oil slurry. Care must be used with these LCPs since they easily degrade and become rancid. The completed oil slurry is held under agitation until it is blended with the other slurries, preferably for a period of no longer than about twelve hours.

A protein in water slurry is prepared by first heating water to an appropriate elevated temperature with agitation. The protein source is then added to the water with agitation. Typically this protein source is intact or hydrolyzed milk proteins (e.g. whey, casein), intact or hydrolyzed vegetable proteins (e.g. soy), free amino acids and mixtures thereof. In general, any known source of amino nitrogen can be used in this invention. The completed protein slurry is held under agitation at elevated temperature until it is blended with the other slurries, preferably for a period no longer than about two hours. As an alternative, some protein may be mixed in a protein-in-fat emulsion rather than protein-in-water.

The protein in water and carbohydrate/mineral slurries are blended together with agitation and the resultant blended slurry is maintained at an elevated temperature. After a brief delay (e.g. a few minutes), the oil slurry is added to the blended slurry from the preceding step with agitation. As an alternative to addition to the oil blend, the LCP oils can be added directly to the blend resulting from combining the protein, carbohydrate/mineral and oil slurries.

After sufficient agitation to thoroughly combine all constituents, the pH of the completed blend is adjusted to the desired range. The blended slurry is then subjected to deaeration, ultra-high temperature heat treatment, emulsification and homogenization, then is cooled to refrigerated temperature. Preferably, after the above steps have been completed, appropriate analytical testing for quality control is conducted. Based on the analytical results of the quality control tests, and appropriate amount of water is added to the batch with agitation for dilution.

A vitamin solution, containing water soluble vitamins and trace minerals (including sodium selenate), is prepared and added to the processed slurry blend with agitation. A separate solution containing nucleotides is prepared and also added to the processed blended slurry with agitation.

The pH of the final product may be adjusted again to achieve optimal product stability. The completed product is then filled into the appropriate metal, glass or plastic containers and subjected to terminal sterilization using conventional technology. Alternatively, the liquid product can be sterilized aseptically and filled into plastic containers.

2.5.2 Powder Products

A carbohydrate/mineral slurry is prepared as was described above for liquid product manufacture.

An oil slurry is prepared as was described above for liquid product manufacture with the following exceptions: 1) Emulsifiers (mono, diglycerides, lecithin) and stabilizers (carrageenan) typically are not added to powder, 2) In addition to the beta carotene, other antioxidants, such as mixed tocopherols and ascorbyl palmitate, can be added to help maintain the oxidative quality of the product during any subsequent spray drying process, and 3) The specific LCPs important to this invention are added after mixing the slurries, rather than to the oil slurry.

A protein in water slurry is prepared as was described above for liquid product manufacture.

The carbohydrate/mineral slurry, protein in water slurry and oil slurry are blended together in a similar manner as described for liquid product manufacture. After pH adjustment of the completed blend, LCPs are then added to the blended slurry with agitation. Desirably, the LCPs are slowly metered into the product as the blend passes through a conduit at a constant rate just prior to homogenization (in-line blending).

After deaeration, ultra-high temperature heat treatment, emulsification and homogenization, the processed blend may be evaporated to increase the solids level of the blend to facilitate more efficient spray drying. The blend then passes through a preheater and a high pressure pump and is spray dryed using conventional spray drying technology. The spray dryed powder may be agglomerated, and then is packaged into metal or plastic cans or foil/laminate pouches under vacuum, nitrogen, or other inert environment.

Variations on any of these manufacturing processes are known to or will be readily apparent to those skilled in the art. It is not intended that the invention be limited to any particular process of manufacture. The full text of all US patents mentioned herein is incorporated by reference.

2.5.3 Pharmaceutical Dosage Forms

Pharmaceutical dosage forms may be useful for both drug and dietary supplement forms. They are well known to those skilled in the art, and include tablets, capsules, pills, powders, and other forms. Methodologies for making each of these dosage forms is well known and, except as noted in an earlier section, will not be repeated here.

### EXAMPLE

3.1 Experimental Design

The plan was to artificially rear rat pups on different n-6 and n-3 formulas and then assess food intake after weaning them onto a semi-solid food. One group of rats was reared and tested in February and the other in April 2002. The intent was to combine the February and April results, however methodological problems (described below) limited the reliability of some of the results from February. The April dataset is reliable and complete. The artificial rearings and food intake studies took place at the University of California, Los Angeles, in Dr. John Edmond's laboratory, kindly under the care of Rose Korsak, who were both blind to the composition of the different rat milk formulas and feeding groups.

3.1.1 Basis for Experimental Design

A 2×2 factorial design using a neonatal gastrostomy reared formula fed rat model was used. Previous research has shown in formula fed rats (Ward et al, 1998 and 1999) and piglets (de la Presa Owens and Innis, 1999 and 2000) that brain fatty acid levels of arachidonic acid (AA) and docosahexaenoic acid (DHA) vary with different dietary levels of AA and DHA with (Ward et al, 1998 and 1999; de la Presa Owens and Innis, 1999) or without (de la Presa Owens and Innis, 2000) adequate levels of their precursors, linoleic acid and linolenic acid, respectively. The dietary levels of AA and DHA in the present study were chosen based on published data from Ward et al (1998) and Wainwright et al (1999) who studied similar levels of AA and DHA using the same gastronomy reared rat model. The levels of AA and DHA studied were 0% and 2.5% total fatty acids alone or in combination; a fourth group was fed no AA or DHA (Table 3.1 and Table 3.2). This phase of the study is the AA and DHA (feeding) rearing phase.

TABLE 3.1
Two-by-two factorial design.

Arachidonic Acid
(20:4n − 6; AA)

| | | |
|---|---|---|
| Docosahexaenoic Acid | 0.0% | 2.5% |
| (22:6n − 3; DHA) | 2.5% | 2.5% |

Fatty acid percentages are expressed as % total fatty acids. Design shown corresponds to No AA, No DHA; No AA, + DHA; +AA, No DHA; +AA, +DHA rat milk formula groups (see Table 3.2).

TABLE 3.2
Experimental groups.

| Formula Groups | AA | DHA |
|---|---|---|
| No AA, No DHA | 0.0% | 0.0% |
| No AA, +DHA | 0.0% | 2.5% |
| +AA, no DHA | 2.5% | 0.0% |
| +AA, +DHA | 2.5% | 2.5% |

The four different rat milk formula groups for the AA and DHA rearing phase are shown. Fatty acids are expressed as % total fatty acids.
AA, arachidonic acid (20:4n − 6);
DHA, docosahexaenoic acid (22:6n − 3).

The base formula was designed to contain marginally adequate levels of linoleic acid and undetectable levels of α-linolenic acid as has been used in studies of rats (Wainwright et al, 1999) and piglets (de la Presa Owens and Innis, 1999 and 2000) to maximize differences in brain levels of AA and DHA among the four experimental groups of rats.

3.1.2 Reference Groups

In addition to the four rat milk formula groups, two reference groups were also studied. One reference group was a normal suckling group in which the rat pups remained with the dam until day 20 when brain tissue was obtained. The normal suckling rats were not included in the food intake phase of the experiment. A second reference group of suckling rats served as an experimental design reference group. At the start of the food intake phase of the experiment, rat pups were removed from the dam and included in the feeding measurements on days 19 and 20. These rats were not weaned or introduced to the mash diet before the initiation of the food intake phase.

3.1.3 Statistical Analyses

Data results were analyzed using SAS/Stat software, version 8.2 (SAS® Institute, Inc., Cary, N.C.). Main effects were assessed for AA and DHA using a two-way, no interaction model. This allowed for comparisons between formulas containing AA and formulas containing DHA. It also gave more power to the statistical analyses by increasing the number of animals per group. The suckling reference groups were each compared to the other groups using a one-way analysis of variance which was adjusted for sample size, but not for multiple comparisons. The level of significance was set at 0.05. The number of animals per group was chosen to be between 8 and 16.

3.2 Rearing Phase

3.2.1 Artificial Rearing Procedure

Pregnant Sprague-Dawley rats were obtained from Charles River Laboratories (Wilmington, Mass.) on day 14 of gestation. They were housed under a controlled temperature environment with a 12-hour light/dark cycle. Rat pups were born on day 21 of gestation within a 24 hour time period. The day of birth was designated day 0.

Male rat pups were removed from dams on postnatal day 6 and artificially reared on rat milk formulas to day 18. This procedure has been described in detail in the literature by Sonnenberg et al, 1982; Smart et al, 1983 and 1984; and Auestad et al, 1989. Similar procedures have been also been described by Ward et al, 1998, and Wainwright et al, 1999. On postnatal day 6, rat pups were randomly assigned to one of the four experimental rat milk formula groups (Table 3.2). The rat pups were lightly anesthetized, fitted with an intragastric cannula, and placed individually in pint-sized plastic containers free floating in a waterbath maintained at 36±2° C. The cannulae for individual rat pups were each connected to syringes filled with one of the four experimental rat milk formulas using polyethylene tubing. The rat pups were fed by intermittent, intragastric infusion, from day 6 to day 18. The formula was delivered to the rat pups for 20 or 30 min each hour, depending on the age of the rat, using a programmable pump housed in a bench-top refrigerator. The pump settings were modified daily to deliver specific quantities of rat milk formula to the rat pups to support normal growth. The study protocol is shown in Table 3.3.

3.2.2 Rat Milk Formula Composition

Rat milk formulas were prepared as described in the literature (Auestad et al, 1989; Ward et al, 1998) except that the protein source was whey and casein powders (kindly provided by Ross Products Division of Abbott Laboratories). Briefly, a premilk base consisting of casein, whey, and water was prepared first. Then, a fat blend (see Table 3.4), lactose, minerals, vitamins, and additional nutrients as found in rat milk were added to the premilk base and mixed using a Polytron homogenizer (see Table 3.5). The fat blends used in preparation of the rat milk were formulated to provide marginal amounts of linoleic acid, linolenic acid, and different amounts of AA and DHA.

TABLE 3.3
Calorie sources during AA and DHA rearing phase and food intake
phase of experiments.

| | Postnatal Age, day | | | | | |
|---|---|---|---|---|---|---|
| | 6-15 | 16 | 17 | 18 | 19 | 20 |
| Caloric Intake, % of caloric needs as: | | | | | | |
| Rat Milk Formula (AA and DHA | 100 | 80 | 80 | 20 | 0 | 0 |
| Rearing Phase) | | | | | | |
| Mash | 0 | 20 | 20 | 20 | | |
| Food Intake Phase, % of diet (ad lib) | | | | | | |
| Mash | | | | | 100 | 100 |

Feeding protocol for the four rat milk formula groups. Experimental rat milk formula was randomly assigned on postnatal day 6 and fed through day 18. The rat milk formula contained no AA or DHA; no AA, 2.5% DHA; 2.5% AA, no DHA; or 2.5% AA, 2.5% DHA. Mash was a semi-solid food and was introduced to all four groups on day 16 and fed exclusively during the food intake phase. The mash met the AIN-93 recommendations for nutrients and contained no AA or DHA.

TABLE 3.4
Fatty acid composition of the fat blends used in the
preparation of experimental rat milk formulas.

| | No AA | | +AA | |
|---|---|---|---|---|
| | No DHA | +DHA | No DHA | +DHA |
| Fat Blend, % of total oils | | | | |
| Coconut oil | 67.5 | 60.0 | 60.1 | 52.6 |
| MCT oil¹ | 32.5 | 27.5 | 27.4 | 22.6 |
| AA oil² | 0.0 | 0.0 | 12.5 | 12.4 |
| DHA oil³ | 0.0 | 12.5 | 0.0 | 12.4 |
| Fatty Acids, % total fatty | | | | |
| acids⁴ | | | | |
| C8:0 | 28.1 | 14.7 | 14.3 | 0.0 |
| C10:0 | 20.1 | 10.5 | 10.1 | 0.2 |
| C12:0 | 27.7 | 15.1 | 13.9 | 0.9 |
| C14:0 | 10.7 | 12.0 | 6.1 | 7.4 |
| C16:0 | 5.9 | 11.1 | 9.9 | 15.4 |
| C18:0 | 1.7 | 1.3 | 6.5 | 6.3 |
| C22:0 | 0.0 | 0.1 | 0.8 | 1.0 |
| C24:0 | 0.0 | 0.1 | 0.9 | 0.9 |
| Sum Saturated | 32.1 | 64.8 | 62.3 | 94.2 |
| C16:1 | 0.0 | 0.6 | 0.1 | 0.7 |
| C18:1 | 4.0 | 12.9 | 8.3 | 17.6 |
| Sum Unsaturated | 4.0 | 13.4 | 8.4 | 18.2 |
| C18:2n − 6 | 1.1 | 0.9 | 4.2 | 4.0 |
| C18:3n − 6 | 0.0 | 0.0 | 1.7 | 1.7 |
| C20:3n − 6 | 0.0 | 0.0 | 1.8 | 1.8 |
| C20:4n − 6 (AA) | 0.1 | 0.0 | 19.9 | 20.1 |
| Sum n − 6 | 1.2 | 0.9 | 27.5 | 27.6 |
| C22:6n − 3 (DHA) | 0.0 | 20.4 | 0.0 | 20.3 |
| Sum n − 3 | 0.0 | 20.4 | 0.0 | 20.3 |

Results are expressed as % total fatty acids.
AA, arachidonic acid or C20:4n − 6;
DHA, docosahexaenoic acid or C22:6n − 3;
¹MCT oil, medium chain triglyceride oil.
² AA oil, ARASCO ™ and
³DHA oil, DHASCO ™ (Martek Biosciences Corp., Columbia, MD), approximately 20% AA and DHA, respectively.
⁴Fatty acids present in concentrations less than 0.5% total fatty acids are not shown.

TABLE 3.5
Ingredients in the rat milk formula.

| INGREDIENT | g/2.5 L |
|---|---|
| PROTEIN: | |
| Casein | 157.5 |
| Whey | 105.8 |
| Water | 1987 |
| Amino Acid Mix | 2.425 |
| CARBOHYDRATE: | |
| Lactose | 87.5 |
| FAT BLEND (see Table 3.4) | 350.0 |
| MINERALS: | |
| Calcium Carbonate | 15.08 |
| Calcium Gluconate | 3.413 |
| Calcium Chloride | 6.95 |
| Non-Calcium Mineral Mix (with | 15.1 |
| Iron) | |
| Copper Sulfate Solution¹ | 0.0749 |
| Zinc Sulfate Solution² | 0.2845 |
| VITAMINS: | |
| Vitamin Mix (Teklad) | 10.0 |
| Vitamin Mix (Supplementary) | 1.375 |
| OTHER: | |
| Carnitine | 0.1 |
| Creatine | 0.175 |
| Ethanolamine | 0.0855 |

Ingredients are listed in gram per 2.5 liters.
¹Copper sulfate solution was 30.9 g CuSO₄.5H₂O/L H₂O;
²Zinc sulfate solution was 379.3 g ZnSO₄.7H₂O/L H₂O (as described in Auestad et al, 1989).

The fatty acid composition of the rat milk formulas was determined by gas chromatographic analysis and results are shown in Tables 3.6. The target percentages of the fatty acids linoleic acid, linolenic acid, AA and DHA, were achieved with concentrations at or near expected targets. There is one formula from the February rearing that appears to be low in AA (1.4% compared to target value of 2.5% total fatty acids). This appears likely due to improper laboratory handling during GC analysis, or other experimental error. It is likely that the result was closer to 2.5% since the exact same fat blends were used to prepare the formulas for both the February and April rearings. The other prepared formulas with added AA ech contained approximately 2.5% AA as expected.

TABLE 3.6
Fatty acid composition of the rat milk formulas for the AA and DHA rearing phase.

| | February Rearing | | | | April Rearing | | | |
|---|---|---|---|---|---|---|---|---|
| | No AA | | +AA | | No AA | | +AA | |
| | No DHA | +DHA | No DHA | +DHA | No DHA | +DHA | No DHA | +DHA |
| C6:0¹ | 0.6 | 0.6 | 0.6 | 0.5 | 0.6 | 0.6 | 0.6 | 0.5 |
| C8:0 | 23.8 | 21.6 | 22.6 | 19.7 | 24.0 | 22.4 | 22.1 | 20.2 |
| C10:0 | 17.2 | 15.7 | 16.4 | 14.7 | 17.0 | 15.9 | 15.7 | 14.6 |
| C12:0 | 29.8 | 29.3 | 29.8 | 27.8 | 31.0 | 29.3 | 29.3 | 27.7 |
| C14:0 | 12.0 | 12.4 | 11.9 | 12.1 | 12.1 | 12.2 | 11.6 | 11.7 |
| C16:0 | 7.8 | 8.4 | 7.9 | 9.2 | 7.0 | 7.7 | 7.6 | 8.2 |
| C18:0 | 2.3 | 2.3 | 2.6 | 3.0 | 2.1 | 2.1 | 2.7 | 2.7 |
| C18:1n-9 | 5.1 | 6.1 | 5.3 | 6.8 | 4.8 | 5.9 | 5.4 | 6.5 |
| C18:2n-6 | 1.3 | 1.3 | 1.5 | 1.7 | 1.3 | 1.2 | 1.7 | 1.6 |
| C20:4n-6 (AA) | 0.0 | 0.0 | 1.4 | 2.4 | 0.0 | 0.0 | 2.5 | 2.5 |
| C22:6n-3 (DHA) | 0.0 | 2.3 | 0.0 | 2.4 | 0.0 | 2.6 | 0.0 | 2.6 |

Results are expressed as % total fatty acids. AA, arachidonic acid or C20:4n-6; DHA, docosahexaenoic acid or C22:6n-3. ¹Fatty acids present at <0.5% total fatty acids are not shown.

3.2.3 Growth Assessment

The artificially reared rat pups were weighed daily. Weights at the beginning and end of the AA and DHA rearing phase as well as weights during the food intake phase will be reported.

3.3 Food Intake Phase

The food mash used in the February experiment was prepared by mixing a fat-free powder meal (Bioserv Inc., Frenchtown, N.J.), a fat blend (coconut oil:MCT oil, 70:30, w/w), and water until the consistency was crumbly. The accuracy of the food intake measurements for the February experiment were questionable due to the consistency of the food mash, therefore, a pelleted food mash with the same nutrient composition was prepared (Research Diets Inc., Princeton, N.J.) for the April experiment. The pellets were extremely dense and hard and there were concerns that the weanling rats may not readily eat the solid pellets. Therefore, the pellets were crushed into powder, mixed with water, and formed into ¼″ to ½″ semi-solid balls which were used in the food intake phase. The nutrient profile of both food mash diets met AIN-93 recommendations (Reeves et al, 1993). Fatty acid analyses were performed on the food mash and results are shown in Table 3.7.

On day 16, the rat pups were introduced to the mash weaning diet. The mash contained 10 g/100 g wet weight as fat, which was a blend of coconut oil:MCT oil (70:30, w/w). All of the rat experimental groups were weaned to the same food mash, which did not contain AA or DHA in order to eliminate potential confounding effects of flavor characteristics from the experimental design. On days 16 and 17, 80-percent of daily caloric requirements were from the assigned experimental rat milk formula, and 20% of calories was from the food mash (Table 3.3). The rat pups consumed all of the wet mash provided within a few minutes.

TABLE 3.7
Fatty acid composition of food mash used for weaning
and during food intake phase of experiments.

| | February¹ | April² |
|---|---|---|
| C6:0³ | 0.6 | 1.3 |
| C8:0 | 23.6 | 27.1 |
| C10:0 | 17.0 | 12.0 |
| C12:0 | 31.4 | 31.8 |
| C14:0 | 12.2 | 12.8 |
| C16:0 | 7.0 | 7.1 |
| C18:0 | 2.1 | 7.1 |
| C18:1n − 9 | 4.7 | 0.9 |
| C18:2n − 6 | 1.3 | 0.2 |
| C20:4n − 6 (AA)* | 0.0 | 0.0 |
| C22:6n − 3 (DHA)* | 0.0 | 0.0 |

Results are expressed in % total fatty acids. The fat blend was coconut oil: MCT oil (70:30, w/w).
¹February food mash was made with fat-free powder to which the fat blend was added along with water to form a crumbly consistency.
²April food mash was made from pellets and contained the same fat blend; the pellets were crushed and formed into ¼″ to ½″ mash balls by adding a small amount of water.
³Fatty acids present in 0.5% or less are not reported except where indicated by ‘*’ for clarity.

Beginning at 5 pm on day 18, the rat pups were calorie restricted with 20% of caloric requirements from the rat milk formula and 20% from the wet mash. The formulas were diluted with water to 20% the initial calorie content. Water intake thus was not restricted to keep the animals properly hydrated. At 9:00 am on day 19, the rat pups were stimulated to urinate and then weighed. The intragastric cannulae were removed, and then rat pups were placed in individual cages containing water bottles and approximately 15 g of ‘crumbly’ mash in ceramic dishes (February experiment) or ‘mash balls’ added directly to the bottom of the cages (April experiment). The cages had clear plastic bottoms and sides, were approximately 8 inches wide×12 inches long, and were enclosed with a wired, slanted top that held a water bottle. Every two hours for eight hours all the remaining mash was weighed to determine the amount of food eaten. Three mash ‘controls’ were included to measure weight loss due to evaporation during the food intake phase. The rats were weighed again at the end of the food intake phase.

The mash was then removed from the cages and the rats fasted for the next 18 hours with free access to water. At 9:00 am on day 20, the rats were again stimulated to urinate, weighed, and placed in their cages with access to water and approximately 15 g of mash. The amount of food eaten and final weights of the rats were determined after 2 hours.

3.4 Tissue Collection

The rat pups were sacrificed by decapitation on day 20 after the food intake phase and final body weights were taken. The brain was removed, weighed, and quickly frozen (within 5 minutes) in liquid nitrogen. Brain tissue was stored in a −70° C. freezer. Blood was collected from the neck stump, mixed with heparin, placed on ice, and centrifuged to ensure adequate phase separation to prepare plasma. Plasma was stored at −70° C.

Brain and plasma samples were shipped overnight on dry ice from UCLA to Ross Products Division of Abbott Labs, Columbus, Ohio, and arrived completely frozen. The shipped samples were inspected for damage and signs of thawing and immediately transferred to a −70° C. freezer for storage until analysis. Plasma samples were obtained but not analyzed as a component of this thesis.

3.5 Lipid Extraction and Analysis

3.5.1 Overview

The fatty acid composition of three lipid fractions in brain was determined. Phospholipid fatty acid methyl esters were determined similar to the methods described by Ward et al (1999). Gas chromatography-mass spectrometry (GC/MS), liquid chromatography-mass spectrometry (LC/MS/MS), as well as HPLC methods for measuring MAG and NAE fatty acids have been described (Berger et al, 2001; Kempe et al, 1996; Fontana et al, 1995; Felder et al, 1996; Wang et al, 2001). However, a less costly and simpler method for measuring these fatty acids in brain tissue was developed.

Total lipid was extracted from rat brains using the Folch extraction method, typical for lipid extraction (Folch et al, 1957). The total lipid extract from each rat brain was separated into neutral lipid and phospholipid fractions using a silica cartridge. The neutral lipid fraction was further separated into MAG and NAE fractions using High Performance Liquid Chromatography (HPLC). Fatty acid composition of the MAG, NAE, and phospholipid fractions was determined using Gas-Liquid Chromatography (GLC) after derivatizing to the corresponding fatty acid methyl esters. The fatty acid composition results correspond to total fatty acids in the membrane phospholipids in brain, and the MAG and NAE fatty acid results represent the concentration of these fatty acyl derivatives in rat brains.

3.5.2 Reagents and Supplies

Arachidonyl ethanolamide and docosatetraeonyl ethanolamide were from Cayman Chemical Co. (Ann Arbor, Mich.). Docosatrieonyl chloride and fatty acid standards were from Nu-Chek Prep, Inc. (Elysan, Minn.). Ethanolamine and boron trifluoride-methanol complex (BF₃) were from Sigma-Aldrich (Milwaukee, Wis.). Dichloromethane, methanol, chloroform, hexane, ethyl acetate, and isopropyl alcohol were from Burdick & Jackson (Muskegon, Mich.), petroleum ether was from Mallinckrodt (Paris, Ky.), and formic acid was from J. T. Baker (Phillipsburg, N.J.). All reagents used were of analytical grade. The LHPK Silica Gel Thin-Layer Chromatography plates and filter paper were from Whatman (Clifton, N.J.). Micropipettes, test tubes, and vials were from VWR Scientific (Bridgeport, N.J.). The HPLC column (Chromegasphere SI-60, 4.6×150 mm, 10μ, 60 Å) was from ES Industries (Marlton, N.J.).

3.5.3 Fatty Acid Standards

The GLC fatty acid standard was prepared. Briefly, a representative mixture of fatty acid methyl esters (≧98% purity) was accurately weighed into a tared 100-mL pear-shaped flask in a specific order to ensure proper blending. After all of the fatty acid methyl esters were added and mixed, the flask was weighed for a final weight of the standard. One hundred milligrams of standard were added to ampules, flushed with nitrogen, sealed with a propane flame, and stored in the −20° C. freezer until use.

The GLC stock standard was prepared by quantitatively transferring the contents of one ampule to a 25-mL volumetric flask and diluting to volume with hexane. The GLC working standard was prepared by diluting the GLC stock standard 1:3 (v/v) with hexane and injecting between 1 and 5 μL onto the GLC.

3.5.4 Internal Standards

Two internal standards were needed, monoheptadecanoin for the MAG fraction and docosatrienoyl ethanolamine (22:3n-3 NAE) for the NAE fraction. Monoheptadecanoin was prepared by accurately weighing 100 mg into a 10-mL volumetric flask and diluting to volume with chloroform. Docosatrienoyl ethanolamine was prepared as described by Hanus et al, 1993. Briefly, approximately 100 mg of docosatrienoyl chloride was dissolved in 1 mL of dichloromethane. The mixture was then transferred to a test tube. One mL of ethanolamine solution (20% in dichloromethane) was added to the test tube at 0° C. and flushed with N₂. The test tube was mixed vigorously every 3 minutes for 15 minutes by shaking. Eight mL of dichloromethane was added to bring the volume to 10 mL. The sample was then washed with 5 mL H₂O under N₂ and mixed vigorously. The sample was centrifuged at 2000 rpm at 20° C. for 2 minutes to separate the aqueous and organic layers. The organic (bottom) layer was aspirated into a clean test tube and washed again with 5 mL H₂O under N₂. The sample was centrifuged as described above and the organic layer was aspirated again into a clean test tube. The combined organic layers were evaporated to dryness under N₂. The sample was then reconstituted in 10.0 mL chloroform:methanol (1:1, v/v), blanketed with N₂, capped tightly, and stored at −20° C. The concentration of the resulting docosatrienoyl ethanolamine internal standard was determined by methylation and followed by quantification by GLC.

3.5.5 Sample Extraction

Rat brain samples, stored frozen at −70° C., were separated into the two hemispheres; one half was used for determination of fatty acids in phospholipid, MAG, and NAE fractions and the other half was refrozen at −70° C. The half brain for analysis was transferred to a 50-mL glass centrifuge tube. Eight mL of methanol was added and the sample homogenized using a Polytron Dispersing and Mixing System (Kinematica, Switzerland) until well blended. The homogenizer probe was rinsed with 2 mL methanol added directly into the centrifuge tube. Twenty mL of chloroform was then added to the sample and mixed vigorously by shaking. The sample was left undisturbed at room temperature for at least 1 hour. Known amounts of internal standards, 9.91 μg of monoheptadecanoin and 3.32 μg of docosatrienoyl ethanolamine, were added. Six mL of 0.9% saline was then added and the sample mixed vigorously by shaking. The sample was then centrifuged for 7 minutes at 2000 rpm at 15° C. until the organic and aqueous layers were well separated using a Beckman Allegra™ 6R Centrifuge; Fullerton, Calif.). The chloroform (bottom) layer was aspirated into a clean 30-mL test tube. The sample was then evaporated to dryness under N₂ and was either stored at −20° C. or reconstituted in 500 μL of chloroform.

3.5.6 SEP-PAK Cartridge Purification

Each sample of the reconstituted brain extract was loaded onto a Silica Plus SEP-PAK cartridge (Waters/Millipore, Milford, Mass.) using a disposable glass pipette. The test tube containing the brain extract was rinsed twice with 500 μL of chloroform, which was then loaded onto the cartridge to ensure that all of the extract was transferred to the cartridge. Neutral lipids were eluted with 15 mL chloroform:methanol (99:1, v/v) and phospholipids were eluted with 15 mL of methanol. The neutral lipid eluant was filtered through a syringe filter (Gelman Acrodisc® CR PTFE, 0.45μ or 0.2μ, 25 mm; Ann Arbor, Mich.) attached to the bottom of the SEP-PAK cartridge. Each eluant was collected into test tubes and evaporated to dryness under N₂.

3.5.7 High Performance Liquid Chromatography Fractionation

Each SEP-PAK eluant, after drying down, was resuspended in 125 μL or 300,UL of hexane:isopropyl alcohol (IPA; 90:10, v/v) and injected onto an Hewlett Packard HPLC (Roseville, Calif.) with a Chromegasphere SI-60 column, 4.6×150 mm, 10μ, 60 Å (ES Industries, Marlton, N.J.) and an evaporative light scattering detector (Alitech ELSD, Deerfield, Ill.) for separation of MAGs and NAEs. The mobile phase gradient is shown in Table 3.8 (adapted from Liu et al, 1993).

A solution containing the internal standards tricosanoic acid, monoheptadecanoin, and docosatrienoyl ethanolamine, was injected in triplicate before each HPLC run to confirm retention times for free fatty acids, MAGs, and NAEs. After confirming consistent retention times, the evaporative light scattering detector was disconnected and the HPLC mobile phase line was connected directly to a fraction collector (BioRad, Model 2128; Hercules, Calif.).

TABLE 3.8
HPLC mobile phase gradient for fractionation of fatty
acids, monoacylglycerols, and N-acylethanolamines.

| Time | % of Mobile | Solvent |
|---|---|---|
| (minutes) | Phase Hexane | Mix¹ |
| 0.0 | 98 | 2 |
| 8.0 | 65 | 35 |
| 8.5 | 2 | 98 |
| 15.0 | 2 | 98 |
| 15.1 | 98 | 2 |
| 19.0 | 98 | 2 |

¹Hexane:isopropanol:ethylacetate: 10% formic acid in isopropanol (80:10:10:1, v/v/v/v). Flow rate is 2.0 mL/minute.

Then, 250 μL of the resuspended neutral lipid fraction extract (i.e. chloroform:methanol SEP-PAK elution) was injected onto the HPLC column and fractions corresponding to elution times for MAGs and NAEs were collected. The MAG and NAE fractions collected from the HPLC for each rat brain sample were then evaporated to dryness under N₂.

3.5.8 Methylation Procedure

The MAG and NAE fractions were resuspended in hexane:isopropyl alcohol:ethyl acetate (80:10:10, v/v/v) and transferred to 2-mL amber screw cap vials. The fractions were again evaporated to dryness under a stream of N₂ at room temperature.

The samples containing MAGs, NAEs, and phospholipids were then methylated by addition of excess boron trifluoride-methanol complex, BF₃, under N₂. After capping tightly with teflon-lined caps, the samples were placed on a heating block at 95° C. for 20 minutes. The samples were cooled to room temperature and opened very carefully. The MAG and NAE r4 samples were transferred in methanol to 15 mL test tubes. Then, 2 mL of 0.9% saline and 4 mL hexane were added to the samples and they were mixed vigorously by shaking. For each sample, the hexane layer was removed using disposable glass pipettes, transferred to clean 15 mL test tubes, and evaporated to dryness under N₂. The MAG and NAE methylation hexane extracts were reconstituted in 100 μL of hexane for analysis of the constituent fatty acids by GLC. The dried phospholipid methylation hexane extracts were reconstituted in 10 mL of hexane and diluted 50 μL of reconstituted extract with 150 μL of hexane for analysis of fatty acid composition by GLC.

3.5.9 Gas-Liquid Chromatography

The fatty acid methyl esters were analyzed using a Hewlett Packard 6890 gas-liquid chromatograph (GLC) equipped with a flame ionization detector; and an Omegawax³²⁰ fused silica column coated with polyethylene glycol, 0.32 mm ID×30m, 0.25 mm film thickness (Supelco, Inc.; Bellefonte, Pa.). The gas chromatographic instrument settings were adjusted for optimum signal sensitivity similar to conditions described in Ward et al (1999). Five μL of each sample was injected onto the gas chromatograph using an autosampler (Hewlett Packard 7673A).

Individual fatty acids were identified by co-elution with corresponding fatty acid methyl ester internal standards. Fatty acid levels in the rat brain phospholipid fractions are reported as relative percent of total fatty acids, as is typically reported in the literature. The specific amounts of individual NAE fatty acids in the brain lipid extract were quantified relative to the NAE internal standard, methyl docosatrienoate. Similarly, the amounts of individual MAG fatty acids in the brain lipid extract were quantified relative to the monoglyceride internal standard, methyl heptadecanoate. MAG and NAE corresponding fatty acids are reported as ng/g and μg/g wet weight of rat brain, respectively.

4.1 Study Groups

Two groups of rats were artificially reared one in February 2002 and one in April 2002. The plan was to cannulate a total of 64 rats, thirty-two per rearing and 8 rats per experimental formula group. Several rats died while on the artificial rearing system. The rats that died were replaced with male suckling rats in the February study but were not replaced after postnatal day 7 in the April study. Only rats that were replaced within 24 hours of the first day of cannulation (postnatal day 6) were included in the final datasets. All rats in the final dataset, therefore, were artificially reared from postnatal day 6 through 18, a total of 13 days, so that any dietary fatty acid effects on the fatty acid composition of the brain phospholipid membrane would be consistent across groups.

A total of 74 rats were cannulated and 13 died. Table 4.1 shows the number of rats per experimental formula group per rearing that were cannulated and died. It is noteworthy that more rats died when DHA was not included in the formula than when DHA was in the formula (10 rats vs 3 rats, respectively, p=0.054).

TABLE 4.1
Number of rats cannulated and deaths in the
February and April rearings.

| Rat Milk | No AA | | + AA | |
|---|---|---|---|---|
| Formula Groups¹ | No DHA | +DHA | No DHA | +DHA |
| February Rearing | | | | |
| Cannulated | 11 | 9 | 10 | 9 |
| Died | 3 | 1 | 2 | 1 |
| April Rearing | | | | |
| Cannulated | 9 | 9 | 9 | 8 |
| Died | 3 | 1 | 2 | 0 |
| Combined Rearings | | | | |
| Cannulated | 20 | 18 | 19 | 17 |
| Died | 6 | 2 | 4 | 1 |

¹Rats were fed rat milk formulas containing different amounts of AA and DHA (see Table 3.6) by gastrostomy tube at 100% of calories from postnatal days 6 to 16, 80% of calories on days 17 and 18, and 20% of calories for the last 18 hours before the food intake study. More deaths occurred when DHA was not in the formula (p = 0.054). No other significant associations between formula groups were found.
AA, arachidonic acid;
DHA, docosahexaenoic acid.

4.2 Growth Data

Growth was evaluated with the combined dataset (February and April) and for the primary dataset (April). There were no significant differences in body weight for the rat milk formula groups when the artificial rearing began (postnatal day 6 or 7; data not shown) and when it ended (postnatal day 18) as well as at the end of the food intake experiment (postnatal day 20) (Table 4.2). There were significant differences in body weight (p<0.05) between the suckling reference groups and the other experimental formula groups. The suckling reference feeding group was significantly larger on day 18 than the four formula groups. And the suckling reference normal group was larger on day 20 than the formula groups and the reference feeding group. Significant differences were also found for brain weights. Rats fed the formulas with DHA had slightly, but significantly smaller brain weights than those fed formulas without DHA (p<0.05). All brain weights were within the range of 1.3 g to 1.4 g.

4.3 Food Intake Study

A food intake study during which all rats were given the same mash diet ad lib was initiated on day 19 (Table 4.3). Results are given for the February and April (combined dataset) and for April alone (primary dataset) since the reliability of the food intake measurement was substantially improved between the February and April rearings.

There was a significant main effect of feeding a rat milk formula with AA on food intake after weaning (Table 4.3). Rats previously fed the formulas with AA ate about 13% more mash than those previously fed formulas without AA, irrespective of the DHA level. This effect was seen for the first 2 hours of the food intake phase on day 19 in the combined dataset and the first 2 hours on both day 19 and day 20 in the primary dataset.

There was also a significant main effect of DHA on food intake after weaning. Rats previously fed the formulas with DHA ate about 11% less mash than those previously fed formulas without DHA, irrespective of the AA level. This was seen for the first 2 hours of the food intake phase on day 19 for both the combined dataset and the primary dataset.

TABLE 4.2
Average body and brain weight across experimental formula and suckling groups.

| | | | | | ANOVA Main | | | |
|---|---|---|---|---|---|---|---|---|
| Experimental | No AA | | +AA | | Effects² | | Suckling Reference³ | |
| Formula¹ | No DHA | +DHA | No DHA | +DHA | AA | DHA | Fdg | Normal |
| Body Weight, | | | | | p value | p value | | |
| g | | | | | | | | |
| Artificial | | | | | | | | |
| Rearing Phase | | | | | | | | |
| Day 18 (3 pm) | | | | | | | | |
| Combined⁴ | 43.3 ± 1.6⁶ | 44.6 ± 2.4 | 43.5 ± 2.2 | 43.2 ± 1.9 | >0.1 | >0.1 | 48.2 ± 1.6* | NA |
| Primary⁵ | 44.4 ± 1.4 | 43.9 ± 1.3 | 42.9 ± 2.9 | 43.5 ± 1.2 | >0.1 | >0.1 | 48.2 ± 1.2* | NA |
| Food Intake | | | | | | | | |
| Phase | | | | | | | | |
| Day 20 | | | | | | | | |
| (12 pm) | | | | | | | | |
| Combined | 47.5 ± 1.7 | 48.2 ± 3.0 | 48.0 ± 1.5 | 47.2 ± 2.6 | >0.1 | >0.1 | 47.1 ± 1.5 | 51.2 ± 5.0* |
| Primary | 47.7 ± 1.0 | 47.3 ± 3.1 | 48.0 ± 1.9 | 47.4 ± 2.2 | >0.1 | >0.1 | 47.1 ± 1.5 | 57.5 ± 0.6* |
| Brain Weight, | | | | | | | | |
| g | | | | | | | | |
| Day 20 | | | | | | | | |
| Combined | 1.34 ± 0.05^{c, d} | 1.32 ± 0.04^{d} | 1.37 ± 0.05^{b, c} | 1.34 ± 0.05^{c, d} | 0.056 (+) | 0.035 (−) | 1.42 ± 0.05^{a} | 1.40 ± 0.05^{a, b} |
| Primary | 1.35 ± 0.04^{b, c} | 1.33 ± 0.03^{c} | 1.40 ± 0.05^{a, b} | 1.35 ± 0.04^{c} | 0.097 (+) | 0.026 (−) | 1.42 ± 0.05^{a, b} | 1.46 ± 0.00^{a} |
| ¹Rats were fed rat milk formulas containing different amounts of AA and DHA (see Table 3.6) by gastrostomy tube at 100% of calories from postnatal days 6 to 16, 80% of calories on days 17 and 18, and 20% of calories for the last 18 hours before the food intake study on day 19. A semi-solid mash containing no AA or DHA was fed ad lib at 20% of calories on | | | | | | | | |
| #days 17 and 18 and as the only dietary source for the food intake study. ²Main effects of feeding AA or DHA were determined by ANOVA; (+) indicates main effect was increased weight and (−) indicates main effect was decreased weight. ³Suckling reference groups were normal suckling rats; the feeding (Fdg) study group was abruptly weaned on day 19 immediately prior to the feeding study and served as an experimental design reference group. ⁴The | | | | | | | | |
| #combined datasets (n = 9-15 rats/group) are results from February and April rearings combined. ⁵The primary dataset (n = 5-8 rats/group) are results from the April rearing alone for which fatty acids in different fractions in brain were also determined. There were no significant differences in body weights, except | | | | | | | | |
| #between the suckling and formula groups where indicated by ‘*’, p < 0.05. ^{a, b, c, d}Differences in brain weight were observed as indicated by ‘^{a, b, c,} or ^{d}’ where brain weights with different letters are significantly different from each other (p < 0.05); ⁶ Mean ± SD; AA, arachidonic acid; DHA, docosahexaenoic acid. | | | | | | | | |

TABLE 4.3
Food intake study for combined and primary datasets.

| | | | | | ANOVA Main | | Suckling | |
|---|---|---|---|---|---|---|---|---|
| | No AA | | +AA | | Effects² | | References³ | |
| Rat Milk Formula Groups¹ | No DHA | +DHA | No DHA | +DHA | AA | DHA | Fdg Study | Normal |
| Combined Datasets⁴ | gm of mash eaten/100 g body weight | | | | p-value | p-value | | |
| Day 19, first 2 hr | 10.3 ± 1.1* | 9.8 ± 1.3 | 11.2 ± 1.6 | 10.5 ± 1.0 | 0.025 (+) | 0.025 (−) | na** | na |
| Day 19, total 8 hr | 17.1 ± 1.3 | 16.8 ± 1.7 | 17.7 ± 1.8 | 17.7 ± 1.2 | >0.10 | >0.10 | na | na |
| Day 20, first 2 hr | 13.1 ± 1.2 | 12.3 ± 1.5 | 12.9 ± 2.0 | 13.9 ± 2.1 | >0.10 | >0.10 | na | na |
| Primary Dataset⁵ | | | | | | | | |
| Day 19, first 2 hr | 10.8 ± 1.4 | 9.6 ± 1.6 | 12.3 ± 1.0 | 10.8 ± 0.9 | 0.011 (+) | 0.010 (−) | 6.8 ± 1.0 | na |
| Day 19, total 8 hr | 17.2 ± 1.6 | 16.8 ± 1.8 | 18.4 ± 1.8 | 17.7 ± 1.0 | >0.10 | >0.10 | 12.6 ± 0.8 | na |
| Day 20, first 2 hr | 12.7 ± 1.3 | 12.0 ± 1.2 | 14.0 ± 1.5 | 13.8 ± 2.1 | 0.015 (+) | >0.10 | 12.0 ± 0.8 | na |
| ¹Rats were fed rat milk formulas containing different amounts of AA and DHA (see Table 3.6) by gastrostomy tube at 100% of calories from postnatal days 6 to 16, 80% of calories on days 17 and 18, and 20% of calories for the last 18 hours before the food intake study. A mash containing no AA or DHA was fed ad lib at 20% of calories on days 17 and 18 and as the only dietary source for the food intake study. Food consumption was measured for 8 hours on day | | | | | | | | |
| #19, then rats were fasted overnight after which 2 hr of food consumption was measured. ²Main effects of feeding AA or DHA were determined by ANOVA; (+) indicates main effect was increased food consumption and (−) indicates main effect was reduced food consumption. ³Suckling reference groups were normal suckling rats; the feeding (fdg) study group was abruptly weaned on day 19 immediately prior to the feeding study and served as an experimental | | | | | | | | |
| #design reference group. ⁴The combined datasets (n = 9-15 rats/group) are results from the two rearings combined. ⁵The primary dataset (n = 5-8 rats/group) are results from the second rearing for which fatty acids in different fractions in brain were also determined. * Mean ± SD; ** na, not available; AA, arachidonic acid; DHA, docosahexaenoic acid. | | | | | | | | |

4.4 Phospholipid Fatty Acid Results

Table 4.4 shows results for fatty acid levels in brain phospholipid membranes expressed as % total fatty acids (i.e.g/100 g total fatty acid). The effects of dietary n-6 and n-3 fatty acids on n-6 and n-3 fatty acid composition in brain in this study were similar to that shown previously in the literature (Ward et al, 1999; de la Presa Owens and Innis, 1999). There were no significant differences in saturated fatty acids among the groups. There was a significant main effect of AA on unsaturated fatty acids (C18:1 and C20:1) in brain phospholipids. There were consistent overall effects of dietary AA decreasing and dietary DHA increasing linoleic acid (C 18:2n-6) and C20:3n-6 levels in phospholipids. For other n-6 phospholipid fatty acids there also were consistent overall main effects of dietary AA increasing and dietary DHA decreasing levels of AA and C22:4n-6. There was also a significant main effect of dietary AA decreasing brain phospholipid DHA; likewise, dietary DHA increased brain phospholipid DHA.

4.5 N-ACYLETHANOLAMINE (NAE) Fatty Acid Results

Results for n-6 and n-3 NAEs are shown in Table 4.5 and expressed as ng/g brain. There was a significant main effect of AA increasing 20:4n-6 NAE in brain. There was also a significant main effect of AA increasing total n-6 NAE in brain. No other significant main effects were found. No significant main effects of dietary DHA on n- or n-3 fatty acids were found.

4.6 Monoacylglycerol (MAG) Fatty Acid Results

Results for n-6 and n-3 MAG are shown in Table 4.6 and expressed asug/g brain. There were no significant main effects of dietary AA on n-6 or n-3 MAG. However, there was a significant main effect of DHA increasing 22:6n-3 MAG as well as increasing total n-3 MAG in brain.

4.7 Leptin and Insulin

Postprandial leptin levels in the rats fed the ARA-containing formulas during the formula feeding phase were about 20-30% lower than for rats fed the formulas without ARA. Postprandial leptin levels in the rats fed the DHA-containing formulas were about 5-15% higher than for rats fed the formulas without DHA. No differences in circulating insulin levels were found among the groups.

Correlation Data

In addition to examining the data for main effects of dietary AA and DHA and ANOVA comparisons between the experimental and suckling groups, Spearman correlations were computed to evaluate whether there may be a relationship between specific NAEs and MAGs (including n-6/n-3 ratios) and food intake. Spearman correlation was chosen as it ranks the data and eliminates the weight of potential outliers. Results are shown in Tables 4.7 and 4.8.

Significant positive correlations (r=0.45, p=0.03) were found for the ratio of 20:4n-6 NAE/22:6n-3 NAE levels and food intake during the first 2 hours on day 19 and for cumulative food intake on days 19 and 20 (Table 4.7).

Significant positive correlations were also found between MAG fatty acids and food intake (Table 4.8). There were positive associations between MAG ratios (20:4n-6 MAG/22:6n-3 MAG and sum of n-6 MAG/sum of n-3 MAG) and food intake at the 2 hr measures on days 19- and 20 as well as for cumulative food intake on days 19 and 20 (r=0.42 to 62, p=0.001 to 0.005). There were also trends (p<0.1) for associations between 22:6n-3 MAG, as well as summation of n-3 MAGs, and food intake both on day 19 during the first 2 hours and for cumulative food intake over the entire feeding study (r=−0.39, p=0.06).

TABLE 4.4
Fatty acid levels in rat brain phospholipid membrane across all experimental groups.

| | | | | | ANOVA Main | | | |
|---|---|---|---|---|---|---|---|---|
| Rat Milk Formula | No AA | | AA | | Effects² | | Suckling References³ | |
| Groups¹ | No DHA | +DHA | No DHA | +DHA | AA | DHA | Fdg Study | Normal |
| Fatty Acid | Mean ± SD | | | | p-value | p-value | | |
| Saturated | | | | | | | | |
| C14:0 | 1.1 ± 0.1 | 1.1 ± 0.1 | 1.1 ± 0.1 | 1.1 ± 0.1 | >0.1 | >0.1 | 1.1 ± 0.1 | 1.0 ± 0.1 |
| C16:0 | 16.9 ± 2.4 | 17.7 ± 0.9 | 18.1 ± 1.4 | 17.9 ± 0.9 | >0.1 | >0.1 | 18.3 ± 0.8 | 18.1 ± 0.6 |
| C18:0 | 18.5 ± 0.5 | 18.0 ± 0.4 | 18.4 ± 0.4 | 18.4 ± 0.3 | 0.085 | >0.1 | 18.4 ± 0.4 | 18.2 ± 0.4 |
| C20:0 | 0.3 ± 0.0 | 0.2 ± 0.0 | 0.2 ± 0.0 | 0.2 ± 0.0 | >0.1 | >0.1 | 0.2 ± 0.0 | 0.3 ± 0.0 |
| C22:0 | 0.1 ± 0.1 | 0.1 ± 0.0 | 0.1 ± 0.0 | 0.1 ± 0.0 | >0.1 | >0.1 | 0.1 ± 0.0 | 0.2 ± 0.0 |
| Unsaturated | | | | | | | | |
| C16:1s | 0.7 ± 0.4 | 1.0 ± 0.3 | 0.9 ± 0.3 | 1.0 ± 0.3 | >0.1 | >0.1 | 1.0 ± 0.3 | 0.9 ± 0.3 |
| C18:1s | 15.3 ± 0.9 | 15.2 ± 0.8 | 14.2 ± 0.2 | 13.9 ± 0.8 | <0.001 (−) | >0.1 | 13.6 ± 0.3 | 14.4 ± 1.0 |
| C20:1s | 1.2 ± 0.2 | 1.0 ± 0.2 | 0.9 ± 0.1 | 0.8 ± 0.2 | 0.007 (−) | >0.1 | 0.8 ± 0.1 | 1.0 ± 0.2 |
| C16:4 | 2.8 ± 0.4 | 3.0 ± 0.4 | 2.9 ± 0.3 | 3.0 ± 0.4 | >0.1 | >0.1 | 2.8 ± 0.6 | 2.7 ± 0.4 |
| n-6 Fatty Acids | | | | | | | | |
| C18:2n-6 | 0.6 ± 0.0 | 0.8 ± 0.1 | 0.3 ± 0.0 | 0.3 ± 0.0 | * (−) | * (+) | 0.9 ± 0.0 | 1.1 ± 0.1 |
| C20:2n-6 | 0.1 ± 0.0 | 0.2 ± 0.1 | 0.1 ± 0.0 | 0.1 ± 0.0 | <0.001 (−) | >0.1 | 0.2 ± 0.0 | 0.2 ± 0.1 |
| C20:3n-6 | 0.7 ± 0.0 | 0.9 ± 0.1 | 0.3 ± 0.0 | 0.4 ± 0.0 | * (−) | * (+) | 0.6 ± 0.0 | 0.7 ± 0.1 |
| C20:4n-6 | 14.4 ± 1.0 | 10.2 ± 0.7 | 16.1 ± 0.5 | 14.4 ± 0.7 | * (+) | * (−) | 14.9 ± 0.4 | 14.2 ± 0.9 |
| C22:4n-6 | 4.4 ± 0.3 | 2.2 ± 0.1 | 6.0 ± 0.5 | 4.5 ± 0.2 | * (+) | * (−) | 4.7 ± 0.1 | 4.7 ± 0.2 |
| C22:5n-6 | 2.8 ± 0.5 | 0.9 ± 0.1 | 5.2 ± 0.8 | 1.0 ± 0.1 | * | * (−) | 1.4 ± 0.1 | 1.2 ± 0.1 |
| n-3 Fatty Acids | | | | | | | | |
| C22:5n-3 | 0.2 ± 0.0 | 0.7 ± 0.1 | 0.3 ± 0.0 | 0.2 ± 0.0 | * | * | 0.3 ± 0.0 | 0.4 ± 0.0 |
| C22:6n-3 | 18.4 ± 1.2 | 25.6 ± 0.6 | 13.9 ± 1.1 | 21.6 ± 0.5 | <0.001 (−) | <0.001 (+) | 19.7 ± 0.5 | 19.6 ± 0.6 |
| Results are expressed as % total fatty acids. Phospholipids were separated from neutral lipids by silica cartridge chromatography. ¹Rat milk formula fatty acids are detailed in Table 3.6. ²Main effects of feeding AA or DHA were determined by ANOVA; (+) indicates main effect was increased food consumption, (−) indicates main effect was reduced food | | | | | | | | |
| #consumption, and (*) indicates significant interactions was found, however main effects were also found as noted. ³Suckling reference groups were normal suckling rats; the feeding (fdg) study group was abruptly weaned on day 19 immediately prior to the feeding study and served as an experimental design reference group. | | | | | | | | |

TABLE 4.5
N-acylethanolamine levels in rat brain in experimental formula and suckling groups.

| Rat Milk | | | | | ANOVA Main | | | |
|---|---|---|---|---|---|---|---|---|
| Formula | No AA | | AA | | Effects² | | Suckling References³ | |
| Groups¹ | No DHA | +DHA | No DHA | +DHA | AA | DHA | Fdg Study | Normal |
| | | | | | p-value | p-value | | |
| C20:4n-6 | 42.9 ± 6.95 | 36.4 ± 9.93 | 51.7 ± 10.8 | 55.4 ± 20.4 | 0.008 (+) | >0.1 | 50.0 ± 29.4 | 47.2 ± 11.7 |
| C22:5n-6 | 40.2 ± 12.4 | 33.0 ± 17.4 | 40.0 ± 10.8 | 43.7 ± 25.0 | >0.1 | >0.1 | 36.7 ± 18.3 | 29.9 ± 17.7 |
| Sum n-6 | 83.1 ± 11.5 | 69.4 ± 25.5 | 91.7 ± 19.0 | 99.0 ± 44.8 | 0.027 (+) | >0.1 | 86.7 ± 44.3 | 77.1 ± 26.5 |
| C22:5n-3 | 15.8 ± 14.7 | 41.3 ± 23.0 | 42.3 ± 14.6 | 33.1 ± 18.2 | (*) | (*) | 27.8 ± 16.7 | 33.3 ± 7.72 |
| C22:6n-3 | 39.5 ± 7.57 | 47.1 ± 9.12 | 46.0 ± 9.07 | 48.5 ± 19.0 | >0.1 | >0.1 | 46.7 ± 7.53 | 47.7 ± 13.5 |
| Sum n-3 | 55.2 ± 21.6 | 88.4 ± 29.5 | 88.3 ± 15.7 | 81.6 ± 32.1 | >0.1 | >0.1 | 74.5 ± 20.7 | 81.0 ± 19.2 |
| Results are in ng/g brain weight. Neutral lipids were separated from phospholipids by silica cartridge chromatography and monoglycerides were separated from N-acylethanolamines using a silica column and HPLC fractionation. ¹Rat milk formula fatty acids are detailed in Table 3.6. ²Main effects of feeding AA or DHA were determined by ANOVA; (+) | | | | | | | | |
| #indicates main effect was increased food consumption, (−) indicates main effect was reduced food consumption, and (*) indicates significant interaction found, but no significant main effect. ³Suckling reference groups were normal suckling rats; the feeding (fdg) study group was abruptly weaned on day 19 immediately prior to the feeding study and served as an experimental design reference group. | | | | | | | | |

TABLE 4.6
Monoacylglycerol levels in rat brain in experimental formula and suckling reference groups.

| Rat Milk | | | | | ANOVA Main | | | |
|---|---|---|---|---|---|---|---|---|
| Formula | No AA | | AA | | Effects² | | Suckling References³ | |
| Groups¹ | No DHA | +DHA | No DHA | +DHA | AA | DHA | Fdg Study | Normal |
| | | | | | p-value | p-value | | |
| C20:2n-6 | 0.059 ± 0.005 | 0.125 ± 0.103 | 0.032 ± 0.020 | 0.054 ± 0.038 | 0.067 | >0.1 | 0.107 ± 0.026 | 0.124 ± 0.072 |
| C20:3n-6 | 0.199 ± 0.020 | 0.345 ± 0.101 | 0.126 ± 0.039 | 0.141 ± 0.044 | * | * | 0.212 ± 0.056 | 0.244 ± 0.116 |
| C20:4n-6 | 3.386 ± 0.870 | 3.241 ± 1.034 | 3.495 ± 0.940 | 3.897 ± 1.005 | >0.1 | >0.1 | 3.997 ± 0.989 | 3.471 ± 1.200 |
| C22:5n-6 | 0.145 ± 0.029 | 0.035 ± 0.026 | 0.324 ± 0.097 | 0.030 ± 0.030 | * | * | 0.044 ± 0.042 | 0.078 ± 0.033 |
| Sum n-6 | 3.788 ± 0.889 | 3.746 ± 1.102 | 3.978 ± 1.051 | 4.123 ± 1.045 | >0.1 | >0.1 | 4.359 ± 1.054 | 3.917 ± 1.394 |
| C22:5n-3 | 0.020 ± 0.023 | 0.050 ± 0.036 | 0.027 ± 0.026 | 0.023 ± 0.023 | * | * | 0.052 ± 0.045 | 0.037 ± 0.039 |
| C22:6n-3 | 0.904 ± 0.155 | 1.638 ± 0.512 | 0.788 ± 0.310 | 1.383 ± 0.413 | >0.1 | 0.002 (+) | 1.236 ± 0.435 | 1.260 ± 0.566 |
| Sum n-3 | 0.924 ± 0.177 | 1.688 ± 0.483 | 0.815 ± 0.298 | 1.406 ± 0.398 | >0.1 | 0.001 (+) | 1.287 ± 0.460 | 1.297 ± 0.588 |
| Results are in μg/g brain weight. Neutral lipids were separated from phospholipids by silica cartridge chromatography and monoglycerides were separated from N-acylethanolamines using a silica column and HPLC fractionation. ¹Rat milk formulas are detailed in Table 4.1 legend. ²Main effects of feeding AA or DHA were determined by ANOVA; | | | | | | | | |
| #(+) indicates main effect was increased food consumption, (−) indicates main effect was reduced food consumption, and (*) indicates significant interaction found, however no main effects were found. ³Suckling reference groups were normal suckling rats; the feeding (fdg) study group was abruptly weaned on day 19 immediately prior to the feeding study and served as an experimental design reference group. | | | | | | | | |

TABLE 4.7
Spearman correlation results for NAE levels vs. food intake.

| | Day 19-2 | | | | | |
|---|---|---|---|---|---|---|
| | hours¹ | | | | Total Food | |
| | | p | Day 20-2 hours¹ | | Intake² | |
| | r | value | r | p value | r | p value |
| n-6 NAEs | | | | | | |
| 20:4n-6 NAE | 0.341 | 0.111 | 0.073 | 0.740 | 0.207 | 0.344 |
| Sum n-6 NAE | 0.336 | 0.117 | −0.074 | 0.737 | 0.110 | 0.618 |
| n-3 NAEs | | | | | | |
| 22:6n-3 NAE | −0.95 | 0.667 | −0.160 | 0.466 | −0.238 | 0.274 |
| Sum n-3 NAE | −0.108 | 0.625 | −0.089 | 0.687 | −0.182 | 0.406 |
| Ratios (NAEs) | | | | | | |
| 20:4n-6/22:6n-3 | 0.447 | 0.033 | 0.271 | 0.211 | 0.446 | 0.033 |
| Sum n-6/Sum n-3 | 0.377 | 0.076 | −0.029 | 0.897 | 0.231 | 0.288 |

NAE, N-acylethanolamine; ¹Food intake data from April rearing. ²Total food intake is summation of all food eaten on days 19 and 20 of the April rearing. ³r, correlation. Spearman is a ranked data correlation.

TABLE 4.8
Spearman correlation results for MAG levels vs. food intake.

| | Day 19-2 | | | | | |
|---|---|---|---|---|---|---|
| | hours¹ | | | | Total Food | |
| | | p | Day 20-2 hours¹ | | Intake² | |
| | r | value | r | p value | r | p value |
| n-6 MAGs | | | | | | |
| 20:4n-6 MAG | 0.208 | 0.342 | 0.035 | 0.876 | 0.100 | 0.660 |
| Sum n-6 MAG | 0.221 | 0.310 | 0.018 | 0.936 | 0.094 | 0.670 |
| n-3 MAGs | | | | | | |
| 22:6n-3 MAG | −0.394 | 0.063 | −0.301 | 0.162 | −0.396 | 0.061 |
| Sum n-3 MAG | −0.393 | 0.063 | −0.291 | 0.179 | −0.389 | 0.066 |
| Ratios (MAGs) | | | | | | |
| 20:4n-6/22:6n-3 | 0.615 | 0.002 | 0.457 | 0.029 | 0.570 | 0.005 |
| Sum n-6/Sum n-3 | 0.646 | 0.001 | 0.424 | 0.044 | 0.565 | 0.005 |

MAG, monoacylglycerol; ¹ Food intake data from April rearing. ² Total food intake is summation of all food eaten on days 19 and 20 of the April rearing. ³ r, correlation. Spearman is a ranked data correlation.

5.0 Discussion and Conclusion

This is the first study to show effects of feeding different dietary n-6 and n-3 polyunsaturated fatty acids prior to weaning on food intake after these fatty acids were no longer being fed. Dietary arachidonic acid (AA), regardless of docosahexaenoic acid (DHA) level, fed from postnatal day 6 to 18 resulted in approximately a 13% increase in food consumption following food restriction on days 19 and 20. Likewise dietary DHA, regardless of AA amount, fed from day 6 to 18 resulted in up to a 12% decrease in food consumption following food restriction on postnatal day 19.

Using an artificially reared rat model, we modified the fatty acid composition of the brain phospholipid membrane through inclusion of different dietary n-6 and n-3 fatty acids as has been demonstrated in previous research (Ward et al, 1998 and 1999; Wainwright et al, 1999). The artificially reared rat model is an excellent choice for modifying brain phospholipid composition as the feeding period occurs during a period of rapid brain growth. Our fatty acid results for phospholipid membrane also were consistent with previous studies using similar diets marginally deficient in essential fatty acids (de la Presa Owens and Innis, 1999 and 2000).

We saw significant increases in 20:4n-6 NAE in brain of rats fed formulas with AA (p=0.008). However, the 40% increase was not the same magnitude of increases reported by Berger et al, 2001. Berger and colleagues reported 4-fold increases in 20:4n-6 NAE in piglets fed 0.2% AA (percent total fatty acids). Berger et al also reported between 5 and 9-fold increases in several n-3 NAEs. We did not show any statistically significant increases in n-3 NAE levels, but did see similar results for MAG fatty acids in brain to those reported by Berger et al. We did not show statistically significant differences in any n-6 MAGs, however we did show statistically significant increases in 22:6n-3 MAG as well as the sum of n-3 MAGs. There are several differences in the study design that may explain why our results differ from those reported by Berger. First, we used an artificially reared rat model with 0% and 2.5% levels of AA and/or DHA, whereas Berger et al used a bottle-fed piglet model with 0.2% AA and 0.16% DHA (% total energy). Secondly, our formulas were marginally deficient in essential fatty acids. Berger et al fed adequate levels of linoleic acid and linolenic acid and reported that dietary AA and DHA can only increase the levels of n-6 and n-3 NAEs when adequate essential fatty acids are present. Thirdly, we sacrificed our rats immediately after the last food intake study (i.e. rats were satiated), whereas Berger et al waited 3 to 4 hours after the last formula feeding. Kirkham et al (2002) recently reported differences in NAE and MAG levels in brain during fasting, feeding, and satiation. They found increased levels of 20:4n-6 NAE and MAG after fasting; decreases in 20:4n-6 MAG during eating, and no changes compared to controls during satiation. These differences in study design may explain, at least in part, why the dietary effects of n-6 and n-3 NAE and MAG fatty acid levels were less pronounced than those reported by Berger Our results did not show a direct relationship between dietary AA induced increase in 20:4n-6 NAE and food intake, as one might expect based on the published literature (Williams et al 1999; Hao et al 2000), but rather the present results suggest an association between the ratio of n-6 and n-3 NAE and MAG fatty acids and food intake. 20:4n-6 NAE is the most studied endocannabinoid with respect to appetite. It is plausible, however, that other endocannabinoids in both the n-6 and n-3 families play a role in regulation of appetite. Endocannabinoids with at least 20 carbons and 3 double bonds demonstrate activity at cannabinoid receptors with n-3 endocannabinoids exhibiting different binding affinities than those in the n-6 family (Mechoulam et al, 1998; Kirkham et al, 2002). Interestingly, we found significant positive associations between diet induced increases in the ratio of n-6/n-3 NAEs and food intake, as well as n-6/n-3 MAG ratios and food intake. We also found significant main effects of dietary DHA being associated with decreased food intake. Additionally, correlations between 22:6n-3 MAG (and sum of n-3 MAG) and food intake showed a negative trend (p=0.06), such that as levels of 22:6n-3 MAG (and sum of n-3 MAGs) increased, food consumption decreased. It appears that individual NAE and MAG n-6 and n-3 fatty acids in brain may be less influential on regulation of appetite following a stimulus than the relative amounts of NAE and MAG n-6 and n-3 fatty acids.

Overall, our study produced potentially important findings in relation to central nervous system regulation of appetite. Most importantly, we showed that dietary n-6 and n-3 fatty acids affect food intake. Since the different n-6 and n-3 diets were fed by gastrostomy tube before the food intake studies and all rats were fed the same mash diet during the food intake study, the observed effects cannot be explained by olfactory or other characteristics of the food (mash). There may be other explanations for these observations such as effects of feeding the different diets on release or activity of hormones (e.g. insulin; leptin) and neurotransmitters (e.g. serotonin) known to be involved in the regulation of appetite. However, based on our data it is reasonable to conclude that these observed effects on food consumption may be mediated through changes in the n-6 and n-3 fatty acid composition of the brain phospholipid membrane and consequently in NAE- and MAG-fatty acid levels. The endogenously formed NAEs and MAGs act through the cannabinoid receptor (CB₁). It is well established that increasing 20:4n-6 NAE leads to overeating. The effect of dietary DHA on food intake has not been previously been studied and the association with reduced food intake was unexpected. Other possibilities for dietary fatty acid induced effect on food intake will need to be evaluated, such as responses of leptin, insulin, and other hormones and neurotransmitters, to stimuli known to lead to food consumption (e.g. sleep deprivation) not studied here. The levels of 20:4n-6 NAE and 20:4n-6 MAG levels reported here in satiated rats were very similar to those reported recently in satiated rats by Kirkham et al (2001). Given these similarities, it is reasonable to conclude that the newly developed methodology for quantifying MAGs and NAEs in brain is a viable alternative to the standard GC/MS method.

In conclusion, we demonstrated for the first time that dietary n-6 and n-3 fatty acids affect food intake, possibly through the formation of specific n-6 and n-3 NAEs and MAGs. Additional studies will be necessary to determine more specifically how dietary fatty acids may mediate central nervous system regulation of appetite.

We claim:

1. A method for decreasing the appetite of a mammal comprising enterally administering to said mammal an amount of long-chain n-3 PUFA effective to decrease the appetite of said mammal.

2. The method according to claim 1 wherein said long-chain n-3 PUFA comprises DHA.

3. The method of claim 2 wherein said long-chain n-3 PUFA is administered independent of AA.

4. The method according to claim 1 wherein said long-chain n-3 PUFA is administered during a growth phase prior to or in conjunction with an appetite-impacting stimulus.

5. The method according to claim 1 wherein said long-chain n-3 PUFA is administered to an infant in a daily amount of about 8 to about 396 mg/kg body weight.

6. The method according to claim 1 wherein said long-chain n-3 PUFA is administered to a child or an adult in a daily amount of about 84 to about 15,832 mg.

7. A method for modulating the appetite of a mammal comprising enterally administering to said mammal an amount of long-chain n-3 PUFA and an amount of long-chain n-6 PUFA in relative amounts effective to modulate the appetite of said mammal.

8. The method according to claim 7 wherein said long-chain n-3 PUFA comprises DPA and said long-chain n-6 PUFA comprises AA.

9. The method according to claim 7 wherein said long-chain n-3 PUFA is administered during a growth phase prior to or in conjunction with an appetite-impacting stimulus.

10. The method according to claim 7 wherein said long-chain n-3 PUFA is administered to an infant in a daily amount of about 8 to about 396 mg/kg body weight.

11. The method according to claim 7 wherein said long-chain n-3 PUFA is administered to a child or an adult in a daily amount of about 84 to about 15,832 mg.

12. A method for antagonizing the CB₁ receptor in the brain of a mammal comprising enterally administering to said mammal an amount of long-chain n-3 PUFA effective to antagonize the CB, receptor activity in the brain of said mammal.

13. The method according to claim 12 wherein said long-chain n-3 PUFA comprises DHA.

14. The method of claim 12 wherein said long-chain n-3 PUFA is administered independent of AA.

15. The method according to claim 12 wherein said long-chain n-3 PUFA is administered during a growth phase prior to or in conjunction with an appetite-impacting stimulus.

16. The method according to claim 12 wherein said long-chain n-3 PUFA is administered to an infant in a daily amount of about 8 to about 396 mg/kg body weight.

17. The method according to claim 12 wherein said long-chain n-3 PUFA is administered to a child or an adult in a daily amount of about 84 to about 15,832 mg.

18. A method for decreasing the incidence of obesity or overweight status in a population of mammals comprising enterally administering to at least some members of said population an amount of long-chain n-3 PUFA effective to modulate negatively the appetite of said mammal.

19. The method according to claim 18 wherein said long-chain n-3 PUFA comprises DHA.

20. The method of claim 18 wherein said long-chain n-3 PUFA is administered independent of AA.

21. The method according to claim 18 wherein said long-chain n-3 PUFA is administered during a growth phase prior to or in conjunction with an appetite-impacting stimulus.

22. The method according to claim 18 wherein said long-chain n-3 PUFA is administered to an infant in a daily amount of about 8 to about 396 mg/kg body weight.

23. The method according to claim 18 wherein said long-chain n-3 PUFA is administered to a child or an adult in a daily amount of about 84 to about 15,832 mg.

24. A method for increasing serum leptin levels of a human or other mammal, said method comprising administering to the human or other mammal an effective amount of a long-chain n-3 PUFA to increase postprandial serum leptin levels.

25. The method of claim 24 wherein the long-chain n-3 PUFA comprises DHA.

26. The method of claim 24 wherein the long-chain n-3 PUFA is administered to a child or an adult in a daily amount of from about 84 to about 15,832 mg.

27. A method for reducing the appetite of a human or other mammal, said method comprising administering to the human or other mammal an effective amount of long-chain n-3 PUFA to increase serum leptin levels.

28. The method of claim 27, wherein the n-3 PUFA comprises DHA.

29. The method of claim 27 wherein the long-chain n-3 PUFA is administered to a child or adult in a daily amount of from about 84 to about 15,832 mg.
